(19) United States
(12) Patent Application Publication
(10) Pub. No.: US 2009/0317412 A1
(43) Pub. Date: Dec. 24, 2009

(54) Induction of an immune response against streptococcus pneumoniae polyaccharides

(76) Inventors: Jeffery L. Alexander, San Deigo, CA (US); Alessandro Sette, La Jolla, CA (US); Mark J. Newman, Carlsbad, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/628,265

(22) PCT Filed: Jun. 3, 2005
(86) PCT No.: PCT/US05/19467

§ 371 Date: Nov. 30, 2006

Related U.S. Application Data
(60) Provisional application No. 60/577,103, filed on Jun. 4, 2004.

(51) Int. Cl. A61K 39/09; A61P 31/04

(52) U.S. Cl. 424/185.1

(57) ABSTRACT

The present invention provides conjugates of pan DR binding peptides with Streptococcus pneumoniae polysaccharides, and methods of preventing and treating diseases associated with Streptococcus pneumoniae infection with such conjugates.

### BACKGROUND OF THE INVENTION

It is generally known that the nature of the immune response raised against a particular vaccine antigen is important to the overall effectiveness of the vaccine. In the case of carbohydrate antigens, a large variety of approaches has been explored in attempts to enhance their immunogenicity, including chemical modification (Jennings, et al. in Towards Better Carbohydrate Vaccines Bell and Torrigiani (eds) pp 11-17, J. Wiley & Sons, London, 1987), administration with adjuvants, noncovalent complexing with proteins, covalent attachment to immunogenic protein carriers (Schneerson, et al. in Towards Better Carbohydrate Vaccines, supra pp 307-327), and replacement of the carbohydrate epitope by a protein replica, either peptides synthesized de novo (so-called mimitopes, Geyson, et al. in Towards Better Carbohydrate Vaccines, supra, pp 103-118) or antiidiotypic antibodies (Soederstroem in Towards Better Carbohydrate Vaccines, supra, pp 119-138).

Covalent attachment of carbohydrate antigens to immunogenic T-dependent protein carriers is known (see, e.g., Schneerson, et al., 152:361-376 (1980); Lepow, et al., J. Pediatr. 106:185-189 (1985); Chu, et al., Infect. Immun., 50:245-256 (1983); Marborg et al., Am. Chem. Soc.) 108:5282-5287 (1985); Anderson et al., Infect. Immun.,39:233-238 (1983); Bartoloni, et al., Vaccine 13:463-470 (1995); and Wessels, et al., J. Infect. Dis. 171:879-884 (1995)).

Immunogenic peptides, containing epitopes recognized by T helper cells, have been found to be useful in inducing immune responses. The use of helper peptides to enhance antibody responses against particular determinants is described for instance in Hervas-Stubbs, et al., Vaccine 12:867-871 (1994).

Although allele-specific polymorphic residues that line the peptide binding pockets of MHC alleles tend to endow each allele with the capacity to bind a unique set of peptides, there are many instances in which a given peptide has been shown to bind to more than one MHC allele. This has been best documented in the case of the human DR isotype, in which it has been noted that several DR alleles appear to recognize similar motifs, and independently, several investigators reported degenerate binding and/or recognition of certain epitopes in the context of multiple DR types, leading to the concept that certain peptides might represent “universal” epitopes (Busch, et al., Int. Immunol. 2:443-451 (1990); Panina-Bordignon, et al., Eur. J. Immunol. 19:2237-2242 (1989); Sinigaglia, et al., Nature 336:778-780 (1988); O'Sullivan, et al., J. Immunol. 147:2663-2669 (1991); Roache, et al, J. Immunol. 144:1849-1856 (1991); Hill, et al., J. Immunol. 147:189-197 (1991)). Although, the previously reported peptides do have the capacity to bind to several DR alleles, they are by no means iniversal.

Pan-DR binding peptides have been described in, e.g., WO 95/07707, Alexander, et al., Immunity 1:751-761 (1994) and U.S. Pat. No. 6,413,935. These peptides have been shown to help in the generation of a CTL response against desired antigens.

More than 90 pneumococcal serotypes, immunologically distinguishable by their polysaccharide capsules, can potentially cause disease. (Pneumococcal disease. In: Epidemiology and prevention of vaccine-preventable diseases. 6^{th} ed. Waldorf (MD): Public Health Foundation; 2000. p. 249-63; Kalin M., Thorax 53(3):159-162 (1998); Hedlund J, et al. Clin Infect Dis. 21(4):948-53 (1995)). Although antibiotics have been used successfully to treat pneumococcal infections, increasing antibiotic resistance has complicated disease management (Linares J et al., 1992, Clin Infect Dis 15:99-105; Koornhof H F et al., 1992, Clin Infect Dis 15:84-94; Zhanel G G et al., 1999, Antimicrob Agents Chemother 43:2504-9).

There are at least 40 serogroups, some comprising multiple serotypes that are immunologically cross-reactive. Current pneumococcal vaccine formulations are combination vaccines containing a mixture of the capsular polysaccharides from the more common serotypes and are effective against invasive disease in older children and adults (Fedson D S, Musher D M, Eskola J Pneumococcal vaccine. In: Plotkin S A, Orenstein W A. Ed. VACCINES. 3^{rd} ed. Philadelphia: WB Saunders, 1998:553-607). The 23-valent pneumococcal polysaccharide vaccines have been made available by various manufacturers worldwide and are effective in individuals 2 years of age or older: however, because they elicit a T-cell-independent response, these vaccines are not effective in children younger than 2 years of age (Eskola J, Anttila M. 1999, 18:543-51)

Currently, the only available conjugate pneumococcal vaccine is a seven-valent formulation to a nontoxic diphtheria variant (CRM197) PREVNAR® from Wyeth.

### BRIEF SUMMARY OF THE INVENTION

The present invention provides compositions comprising a mixture of at least two Streptococcus pneumoniae capsular polysaccharides from different Streptococcus pneumoniae serotypes, wherein the capsular polysaccharide from each serotype is conjugated to a separate polypeptide comprising a pan DR binding peptide sequence. In some embodiments, the pan DR binding peptide sequence is independently selected from the formula R1-R2-R3-R4-R5, wherein:
R1 is an amino acid followed by alanine or lysine;

R2 is selected from the group consisting of tyrosine, phenylalanine or cyclohexylalanine;

R3 is 3 or 4 amino acids, wherein each amino acid is independently selected from the group consisting of alanine, isoleucine, serine, glutamic acid and valine;

R4 is selected from the group consisting of threonine-leucine-lysine, lysine-threonine, or tryptophan-threonine-leucine-lysine; and,

R5 consists of 2 to 4 amino acids followed by an amino acid wherein each of the 2 to 4 amino acids is independently selected from the group consisting of alanine, serine, and valine.

The present invention also provides methods of inducing an immune response in a mammal. In some embodiments, the methods comprise administering to the mammal a mixture of at least two Streptococcus pneumoniae capsular polysaccharides from different Streptococcus pneumoniae serotypes, wherein the capsular polysaccharide from each serotype is conjugated to a separate pan DR binding peptide sequence selected from the formula R1-R2-R3-R4-R5, wherein:
R1 is an amino acid followed by alanine or lysine;
R2 is selected from the group consisting of tyrosine, phenylalanine or cyclohexylalanine;
R3 is 3 or 4 amino acids, wherein each amino acid is independently selected from the group consisting of alanine, isoleucine, serine, glutamic acid and valine;
R4 is selected from the group consisting of threonine-leucine-lysine, lysine-threonine, or tryptophan-threonine-leucine-lysine; and,
R5 consists of 2 to 4 amino acids followed by an amino acid wherein each of the 2 to 4 amino acids is independently selected from the group consisting of alanine, serine, and valine.

The present invention also provides methods of making a Streptococcus pneumoniae vaccine. In some embodiments, the method comprises conjugating at least two Streptococcus pneumoniae capsular polysaccharides from different Streptococcus pneumoniae serotypes to two separate polypeptides, each comprising a pan DR binding peptide sequence, wherein the pan DR binding peptide sequence is selected from the formula R1-R2-R3-R4-R5, wherein:
R1 is an amino acid followed by alanine or lysine;
R2 is selected from the group consisting of tyrosine, phenylalanine or cyclohexylalanine;
R3 is 3 or 4 amino acids, wherein each amino acid is independently selected from the group consisting of alanine, isoleucine, serine, glutamic acid and valine;
R4 is selected from the group consisting of threonine-leucine-lysine, lysine-threonine, or tryptophan-threonine-leucine-lysine; and,
R5 consists of 2 to 4 amino acids followed by an amino acid wherein each of the 2 to 4 amino acids is independently selected from the group consisting of alanine, serine, and valine.

In some embodiments, the compositions comprise capsular polysaccharides from at least any five of the following serotypes serotypes: 1, 2, 3, 4, 5, 6B, 7F, 8, 9N, 9V, 10A, 11A, 12F, 14, 15B, 17F, 18C, 19A, 19F, 20, 22F, 23F, 33F, 6A, 7A, 7B, 7C, 9A, 9L, 12A, 13, 15A, 15C, 16F, 18A, 18B, 18F, 19B, 19C, 21, 22A, 23A, 23B, 24F, 25, 27, 29, 31, 34, 35, 38, 45, or 46, wherein each polysaccharide is conjugated to a separate polypeptide comprising the pan DR binding peptide sequence. In some embodiments, the compositions comprise capsular polysaccharides from serotypes 4, 6B, 9V, 14, 18C, 19F, and 23F, wherein each polysaccharide is conjugated to a separate polypeptide comprising the pan DR binding peptide sequence. In some embodiments, the compositions comprise capsular polysaccharides from serotypes 1, 4, 5, 6B, 9V, 14, 18C, 19F, and 23F, wherein each polysaccharide is conjugated to a separate polypeptide comprising the pan DR binding peptide sequence. In some embodiments, the compositions comprise capsular polysaccharides from serotypes 1, 3, 4, 5, 6B, 7F, 9V, 14, 18C, 19F, and 23F, wherein each polysaccharide is conjugated to a separate polypeptide comprising the pan DR binding peptide sequence. In some embodiments, the compositions comprise capsular polysaccharides from serotypes 1, 2, 3, 4, 5, 6B, 7F, 8, 9N, 9V, 10A, 11A, 12F, 14, 15B, 17F, 18C, 19A, 19F, 20, 22F, 23F, and 33F, wherein each polysaccharide is conjugated to a separate polypeptide comprising the pan DR binding peptide sequence.

In some embodiments, the capsular polysaccharide is purified from bacteria of each serotype and conjugated to the polypeptide. In some embodiments, capsular polysaccharide from each serotype is separately conjugated to a polypeptide comprising the pan DR peptide and the resulting conjugates are subsequently combined to form a mixture of conjugates. In some embodiments, capsular polysaccharides from each serotype are combined to form a mixture of polysaccharides and the mixture is subsequently conjugated to polypeptides comprising the pan DR binding peptide.

In some embodiments, the polypeptide comprising the pan DR binding peptide consists of 50 or fewer amino acids. In some embodiments, a polypeptide comprising the pan DR binding peptide consists of 25 or fewer amino acids. In some embodiments, a polypeptide comprising the pan DR binding peptide consists of 15 or fewer amino acids.

In some embodiments, a polypeptide comprising the pan DR binding peptide comprises the amino acid sequence AKXVAAWTLKAAA (SEQ ID NO:5), aKXVAAWTLKAAa, AKFVAAWTLKAAA (SEQ ID NO:6), or aKFVAAWTLKAAa, wherein X is cyclohexylalanine. In some embodiments,a polypeptide comprising the pan DR binding peptide consists of the amino acid sequence AKXVAAWTLKAAA (SEQ ID NO:5), aKXVAAWTLKAAa, AKFVAAWTLKAAA (SEQ ID NO:6), or aKFVAAWTLKAAa, wherein X is cyclohexylalanine.

In some embodiments, the polysaccharide and the polypeptide are linked via a linker.

### Definitions

An “oligopeptide” or “peptide” as used herein refers to a chain of at least four amino acid or amino acid mimetics, e.g., at least six, e.g., eight to ten, e.g., eleven to fourteen residues, e.g., fewer than about fifty residues, e.g., fewer than about twenty-five, e.g., fewer than fifteen, e.g., eight to fourteen residues. The oligopeptides or peptides can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described.

When referring to an amino acid residue in a peptide, oligopeptide or protein, the terms “amino acid residue”, “amino acid” and “residue” are used interchangeably and, as used herein, mean an amino acid or amino acid mimetic joined covalently to at least one other amino acid or amino acid mimetic through an amide bond or amide bond mimetic.

As used herein, the term “amino acid”, when unqualified, refers to an “L-amino acid” or L-amino acid mimetic.

Although the peptides may be substantially free of other naturally occurring proteins and fragments thereof, in some embodiments the peptides can be synthetically conjugated to other peptides or polypeptides, e.g. chemically conjugated or recombinantly fused.

As used herein, the term “biological activity” means the ability to bind an appropriate MHC molecule and, in the case of peptides useful for stimulating immune responses, induce a T helper response, which in turn helps to induce an immune response against a target immunogen or immunogen mimetic. In the case of peptides useful for stimulating antibody responses, the peptide will induce a T helper response, which in turn helps induce a humoral response against the target immunogen.

A “pan DR-binding peptide” (also termed a “PADRE® peptide”) of the invention is a peptide capable of binding at least about 7 of the 12 most common DR alleles (DR1, 2w2b, 2w2a, 3, 4w4, 4w14, 5, 7, 52a, 52b, 52c, and 53).

The terms “immunogen” and “antigen” are used interchangeably and mean any compound to which a cellular or humoral immune response is to be directed against.

As used herein, the term “antigenic determinant” is any structure that can elicit, facilitate, or be induced to produce an immune response, for example carbohydrate epitopes, lipids, proteins, peptides, or combinations thereof.

A “CTL epitope” of the present invention is one derived from selected epitopic regions of potential target antigens, such as Streptococcus-derived protein antigens.

A “humoral response” of the present invention is an antibody-mediated immune response directed towards various regions of an antigenic determinant. One of skill will recognize that a humoral response may also be induced against a pan DR binding peptide, wherein the pan DR binding peptide would also be included with the determinant. Thus the elicited immune response may be against both the antibody inducing determinant and the pan DR binding peptide.

A “carbohydrate epitope” as used herein refers to a carbohydrate structure, present as a glycoconjugate, e.g., glycoprotein, glycopeptide, glycolipid, and the like, or a polysaccharide, oligosaccharide, or monosaccharide against which an immune response is desired. The carbohydrate epitope may induce a wide range of immune responses. One of skill will recognize that various carbohydrate structures exemplified herein can be variously modified according to standard methods, without adversely affecting antigenicity. For instance, the monosaccharide units of the saccharide may be variously substituted or even replaced with small organic molecules, which serve as mimetics for the monosaccharide.

“Serotype” as used herein refers to what are generally known in the art as either serotypes or serogroups. The serotypes described herein are referred to by their Danish designation. The Pneumococcal type corresponding to the Danish designation is well established. For example, the following table provides a partial conversion list.

| Pneumococcal | Danish |
|---|---|
| Type | Designation |
| 1 | 1 |
| 2 | 2 |
| 3 | 3 |
| 4 | 4 |
| 5 | 5 |
| 8 | 8 |
| 9 | 9N |
| 12 | 12F |
| 14 | 14 |
| 17 | 17F |
| 19 | 19F |
| 20 | 20 |
| 22 | 22F |
| 23 | 23F |
| 25 | 25 |
| 26 | 6B |
| 34 | 10A |
| 43 | 11A |
| 51 | 7F |
| 54 | 15B |
| 56 | 18C |
| 57 | 19A |
| 68 | 9V |
| 70 | 33F |

The phrases “isolated” or biologically pure” refer to material which is substantially or essentially free from components which normally accompany it as found in its native state. Thus, the peptides of the present invention do not contain materials normally associated with their in situ environment, e.g., MHC Class I molecules with antigen presenting cells. Even if a protein has been isolated to a homogeneous or dominant band in an electrophoretic gel, there are trace contaminants in the range of 5-10% of native protein which co-purify with the desired protein. Isolated peptides of this invention do not contain such endogenous co-purified protein. Similarly, isolated polysaccharides do not comprise more than trace amounts of proteins or other cell components from the bacteria from which they are derived.

A “linker” as used herein is any compound used to provide covalent linkage and spacing between two functional groups (e.g., a pan DR binding peptide and a desired immunogen). Typically, the linker comprises neutral molecules, such as aliphatic carbon chains, amino acids or amino acid mimetics, which are substantially uncharged under physiological conditions and may have linear or branched side chains. In some cases, the linker may, itself, be immunogenic, although non-therapeutically directed. Various linkers useful in the invention are described in more detail, below. Additionally, the verbs “link” and “conjugate” are used interchangeably herein and refer to covalent attachment of two or more species.

A “T helper peptide” as used herein refers to a peptide recognized by the T cell receptor of T helper cells. The pan DR binding peptides of the present invention are T helper peptides.

A “capsular polysaccharide from a Streptococcus pneumoniae serotype” refers to polysaccharides (or at least an epitope thereof) purified from the capsule of Streptococcus pneumoniae bacteria of that serotype or a synthetically manufactured polysaccharide having the same structure (or at least an epitope thereof) as the native polysaccharide of that serotype.

“Conjugating capsular polysaccharides from two serotypes to separate peptides” refers to a process that results in conjugation of a capsular polysaccharide from a first serotype to a first peptide and conjugation of a capsular polysaccharide from a second serotype to a second peptide. The first and second peptides may have the same amino acid sequence, or may have different sequences.

### DETAILED DESCRIPTION OF THE INVENTION

The compositions of the invention generally comprise two components, i.e., a pan DR binding peptide and one or more bacterial capsular polysaccharides. Generally, the pan DR binding peptide, or a polypeptide comprising the peptide sequence, is conjugated to a bacterial capsular polysaccharide, e.g., from a S. pneumoniae serotype. The invention further provides compositions comprising mixtures of such conjugates so that polysaccharides from at least two serotypes are combined in the composition (e.g., a capsular polysaccharide from one serotype conjugated to one pan DR binding peptide mixed with a capsular polysaccharide from a second serotype conjugated to a second (same or different) pan DR binding peptide). The present invention is useful for eliciting an immune response, typically, a humoral response, to antigenic determinants of a carbohydrate immunogen, and in particular Streptococcus pnemoniae capsular polysaccharides.

The nomenclature used to describe peptide compounds follows the conventional practice wherein the amino group is presented to the left (the N-terminus) and the carboxyl group to the right (the C-terminus) of each amino acid residue. In the amino acid structure formulae, each residue is generally represented by standard three letter or single letter designations. The L-form of an amino acid residue is represented by a capital single letter or a capital first letter of a three-letter symbol, and the D-form for those amino acids having D-forms is represented by a lower case single letter or a lower case three letter symbol. Glycine has no asymmetric carbon atom and is simply referred to as “Gly” or G.

The nomenclature used to describe carbohydrates includes the following abbreviations: Ara=arabinosyl; Fru=fructosyl; Fuc=fucosyl; Gal=galactosyl; GalNAc=N-acetylgalacto; Glc=glucosyl; GlcNAc=N-acetylgluco; Man=mannosyl; and NeuAc=sialyl (N-acetylneuraminyl).

Carbohydrates are considered to have a reducing end and a non-reducing end, whether or not the saccharide at the reducing end is in fact a reducing sugar.

All carbohydrates herein are described with the name or abbreviation for the non-reducing saccharide (e.g., Gal), followed by the configuration of the glycosidic bond (α or β), the ring bond, the ring position of the reducing saccharide involved in the bond, and then the name or abbreviation of the reducing saccharide (e.g., GlcNAc). The linkage between two sugars may be expressed, for example, as 2,3, 2→3, or (2,3). Each saccharide is a pyranose.

### 1. Pan DR-Binding Peptides

The present invention provides methods useful for identification of modifications to a starting peptide which broaden its specificity. For instance, International Application Publication No WO 92/02543 describes methods suitable for identifying peptides capable of binding DR molecules. WO 92/02543 describes the use of hemagglutinin from the influenza virus (“HA”), as the source of peptides specifically reacting with HLA-DR. Portions of the protein are screened for reactivity to provide sequences which bind the appropriate DR molecule, such as DR1, DR4w4 or DR4w14.

Once an immunogen or peptide thereof which binds to the selected MHC molecule is identified, a “core binding region” of the antigen or peptide may be determined by synthesizing overlapping peptides, and/or employing N-terminal or C-terminal deletions (truncations) or additions. In the determination of a core binding region and critical contact residues, a series of peptides with single amino acid substitutions may be employed to determine the effect of electrostatic charge, hydrophobicity, etc. on binding.

Within the core region, “critical contact sites,” i.e., those residues (or their functional equivalents) which must be present in the peptide so as to retain the ability to bind an MHC molecule and inhibit the presentation to the T cell, may be identified by single amino acid substitutions, deletions, or insertions. In addition, one may also carry out a systematic scan with a specific amino acid (e.g., Ala) to probe the contributions made by the side chains of critical contact residues.

The peptides of the invention are relatively insensitive to single amino acid substitutions with neutral amino acids, except at essential MHC and TCR contact sites, and have been found to tolerate multiple substitutions. Exemplary multiple substitutions are small, relatively neutral moieties such as Ala, Gly, Pro, or similar residues. The number and types of residues which are substituted or added depend on the spacing necessary between essential contact points and certain functional attributes which are sought (e.g., hydrophobicity versus hydrophilicity). Increased binding affinity for an MHC molecule may also be achieved by such substitutions, compared to the affinity of the parent peptide. In any event, such “spacer” substitutions should employ amino acid residues or other molecular fragments chosen to avoid, for example, steric and charge interference which might disrupt binding.

The effect of single amino acid substitutions may also be probed using D-amino acids. Such substitutions may be made using well known peptide synthesis procedures, as described in e.g., Merrifield, Science 232:341-347 (1986), Barany and Merrifield, THE PEPTIDES, Gross and Meienhofer, eds. (N.Y., Academic Press), pp. 1-284 (1979); and Stewart and Young, SOLID PHASE PEPTIDE SYNTHESIS, (Rockford, Ill., Pierce), 2d Ed. (1984).

The peptides employed in the subject invention need not be identical to peptides disclosed herein, so long as the subject compounds are able to bind to the appropriate MHC molecules or provide for humoral or cytotoxic T lymphocytic activity against the target immunogen. Thus, one of skill will recognize that a number of conservative substitutions can be made without substantially affecting the activity of the peptide. Conservative substitutions in which an amino acid residue is replaced with another which is biologically and/or chemically similar, e.g., one hydrophobic residue for another, or one polar residue for another. The substitutions include combinations such as Gly, Ala; Val, Ile, Leu, Met; Asp, Glu; Asn, Gln; Ser, Thr; Lys, Arg; and Phe, Tyr.

In addition, the peptide or oligopeptide sequences can differ from the natural sequence by being modified by terminal-NH₂ acylation, e.g., by alkanoyl (C₁-C₂₀) or thioglycolyl acetylation, terminal-carboxy amidation, e.g., ammonia, methylamine, etc. In some instances these modifications may provide sites for linking to a support or other molecule.

Another approach may be used in which anchor residues that contain side chains critical for the binding to the MHC are inserted into a poly-alanine peptide of 13 residues. This approach has been used by Jardetzky, et al., Nature 353:326-329 (1990). They demonstrated that a polyalanine peptide which was modified with a single dominant MHC contact residue (Tyr) endowed the peptide with high affinity binding capacity for DR1. Instead of using tyrosine as the main MHC contact residue, cyclohexylalanine or phenylalanine can also be utilized. These residues are interchangeable with Tyr with respect to a peptide's capacity to bind those DR alleles capable of high affinity binding of the HA peptide, and furthermore also allow binding to MHC molecules that contain a G→V substitution at residue 86 in the DR β chain. This change affects the binding specificity of the B binding pocket in class II MHC such that tyrosine is no longer capable of effective binding, whereas cyclohexylalanine, as well as phenylalanine, can bind.

The biological activity of the peptides identified above may be assayed in a variety of systems. For example, CD4⁺ cell activity in response to immunization with the peptides may be used, e.g., as described in the Examples. Alternatively, the ability to inhibit antigen-specific T cell activation is tested. In one exemplary protocol, an excess of peptide is incubated with an antigen-presenting cell of known MHC expression, (e.g., DR1) and a T cell clone of known antigen specificity (e.g., tetanus toxin 830-843) and MHC restriction (again, DR1), and the immunogenic peptide itself (i.e., tetanus toxin 830-843). The assay culture is incubated for a sufficient time for T cell proliferation, such as four days, and proliferation is then measured using standard procedures, such as pulsing with [³H]-thymidine during the last 18 hours of incubation. The percent inhibition, compared to the controls which do not receive peptide, is then calculated.

The capacity of peptides to inhibit antigen presentation in an in vitro assay has been correlated to the capacity of the peptide to inhibit an immune response in vivo. In vivo activity may be determined in animal models, for example, by administering an immunogen known to be restricted to the particular MHC molecule recognized by the peptide, and the immunomodulatory peptide. T lymphocytes are subsequently removed from the animal and cultured with a dose range of immunogen. Inhibition of stimulation is measured by conventional means, e.g., pulsing with [³H]-thymidine, and comparing to appropriate controls. See also, Adorini, et al., Nature 334:623-625 (1988), incorporated herein by reference.

A large number of cells with defined MHC molecules, particularly MHC Class II molecules, are known and readily available from, for instance, the American Type Culture Collection (ATCC) (“Catalogue of Cell Lines and Hybridomas,” 6th edition (1988)) Rockville, Md., U.S.A.

An exemplary embodiment of the peptides of the present invention comprises modifications to the N- and C-terminal residues. As will be well understood by the artisan, the N- and C-termini may be modified to alter physical or chemical properties of the peptide, such as, for example, to affect binding, stability, bioavailability, ease of linking, and the like.

Modifications of peptides with various amino acid mimetics or D-amino acids, for instance at the N- or C-termini, are useful for instance, in increasing the stability of the peptide in vivo. Such peptides may be synthesized as “inverso” or “retroinverso” forms, that is, by replacing L-amino acids of a sequence with D-amino acids, or by reversing the sequence of the amino acids and replacing the L-amino acids with D-amino acids. As the D-peptides may be more resistant to peptidases, and therefore may be more stable in serum and tissues compared to their L-peptide counterparts, the stability of D-peptides under physiological conditions may more than compensate for a difference in affinity compared to the corresponding L-peptide. Further, L-amino acid-containing peptides with or without substitutions can be capped with a D-amino acid to inhibit exopeptidase destruction of the immunogenic peptide.

Stability can be assayed in a number of ways. For instance, peptidases and various biological media, such as human plasma and serum, have been used to test stability. See, e.g., Verhoef, et al., Eur. J. Drug Metab. Pharmacokin. 11:291-302 (1986); Walter, et al., Proc. Soc. Exp. Biol. Med. 148:98-103 (1975); Witter, et al., Neuroendocrinology 30:377-381(1980); Verhoef, et al., J. Endocrinology 110:557-562 (1986); Handa, et al., Eur. J. Pharmacol. 70:531-540 (1981); Bizzozero, et al., Eur. J. Biochem. 122:251-258 (1982); Chang, Eur. J. Biochem. 151:217-224 (1985).

Stability may also be increased by introducing D-amino acid residues at the C- and N-termini of the peptide. Previous studies have indicated that the half-life of L-amino acid-containing peptides in vivo and in vitro, when incubated in serun-containing medium, can be extended considerably by rendering the peptides resistant to exopeptidase activity by introducing D-amino acids at the C- and N-termini.

The peptides or analogs of the invention can be modified by altering the order or composition of certain residues, it being readily appreciated that certain amino acid residues essential for biological activity, e.g., those at critical contact sites, may generally not be altered without an adverse effect on biological activity. The non-critical amino acids need not be limited to those naturally occurring in proteins, such as Lα-amino acids, or their D-isomers, but may include non-protein amino acids as well, such as β-γ-δ-amino acids, as well as many derivatives of L-α-amino acids. As discussed, a peptide of the present invention may generally comprise either L-amino acids or D-amino acids, but not D-amino acids within a core binding region. In any sequence described herein, the termini of the peptides can be either in the D- or L-form.

The peptides of the invention can be prepared in a wide variety of ways. Because of their relatively short size, the peptides can be synthesized in solution or on a solid support in accordance with conventional techniques. Various automatic synthesizers are commercially available and can be used in accordance with known protocols. See, e.g., Stewart and Young, Solid Phase Peptide Synthesis, 2d. Ed., Pierce Chemical Co. (1984), supra.

Alternatively, recombinant DNA technology may be employed wherein a nucleotide sequence which encodes an immunogenic peptide of interest is inserted into an expression vector, transformed or transfected into an appropriate host cell and cultivated under conditions suitable for expression. These procedures are generally known in the art, as described generally in Sambrook, et al., Molecular Cloning, A Laboratory Manual, Cold Spring Harbor Press, Cold Spring Harbor, N.Y. (1989). Thus, fusion proteins which comprise one or more peptide sequences of the invention can be used to present the appropriate T cell epitope. It is well-known in the art that, although a peptide comprising one or more D-amino acid residues cannot be produced by recombinant DNA technology, a typically acceptable substitute thereof may be produced by incorporating a DNA sequence that encodes the L-amino acid residue that corresponds to each D-amino acid residue in the original peptide.

As the coding sequence for peptides of the length contemplated herein can be synthesized by chemical techniques, for example, the phosphotriester method of Matteucci, et al., J. Am. Chem. Soc. 103:3185 (1981), modification can be made simply by substituting the appropriate base(s) for those encoding the native peptide sequence. Nucleic acid sequences that encode for appropriate linkers can then be added to the peptide coding sequence and ligated into expression vectors commonly available in the art, and the vectors used to transform suitable hosts to produce the desired fusion protein. A number of such vectors and suitable host systems are now available. For expression of the fusion proteins, the coding sequence will be provided with operably linked start and stop codons, promoter and terminator regions and usually a replication system to provide an expression vector for expression in the desired cellular host. For example, promoter sequences compatible with bacterial hosts are provided in plasmids containing convenient restriction sites for insertion of the desired coding sequence. The resulting expression vectors are transformed into suitable bacterial hosts. Of course, yeast or mammalian cell hosts may also be used, employing suitable vectors and control sequences.

Exemplary pan-DR peptides of the invention include, e.g., oligopeptide of less than about 50 amino acid residues and an antigenic determinant, wherein the oligopeptide and antigenic determinant are optionally covalently attached to each other. The antigenic determinant can be from a bacterium, a virus, a cancer cell, a fungus, or a parasite. When the pan DR binding oligopeptide and the antigenic determinant are covalently attached to each other, they will be either directly linked or attached by means of a linking group.

In one group of embodiments, the pan DR binding peptide is selected from the group consisting of aAXAAAKTAAAAa, aAXAAAATLKAAa, aAXVAAATLKAAa, aAXIAAATLKAAa, aKXVAAWTLKAAa, aKFVAAWTLKAAa, AAXAAAKTAAAAA (SEQ ID NO:1), AAXAAAATLKAAA (SEQ ID NO:2), AAXVAAATLKAAA (SEQ ID NO:3), AAXIAAATLKAAA (SEQ ID NO:4), AKXVAAWTLKAAA (SEQ ID NO:5), and AKFVAAWTLKAAA (SEQ ID NO:6) wherein a is D-alanine, A is L-alanine, X is cyclohexylalanine, K is lysine, T is threonine, L is leucine, V is valine, I is isoleucine, W is tryptophan, and F is phenylalanine. In some embodiments, the pan DR binding peptide is aKXVAAWTLKAAa.

The present invention provides a composition for eliciting an immune response to an immunogenic carbohydrate, the composition comprising a pan DR binding oligopeptide of less than about 50 residues and at least one carbohydrate epitope. In some embodiments, the pan DR binding peptide has the formula R₁-R₂-R₃-R₄-R₅, proceeding from the N-terminus to the C-terminus, wherein R₁ consists of at least 2 residues; R₂ is selected from the group consisting of a cyclohexylalanine residue, a tyrosine residue, a phenylalanine residue and conservative substitutions therefor; R₃ is 3 to 5 amino acid residues; R₄ is selected from the group consisting of threonine-leucine-lysine, lysine-threonine, and tryptophan-threonine-leucine-lysine, and conservative substitutions therefor; and R₅ consists of at least 2 residues. In certain embodiments, each amino acid residue component of a peptide represented by the formula R₁-R₂-R₃-R₄-R₅ can be either a D-amino acid residue or an L-amino acid residue.

The pan DR binding peptides of the invention, in addition to promoting an immune response against a second determinant, can also serve as target immunogens, themselves. Thus, for instance, in the case in which a polypeptide comprising a pan DR binding peptide sequence is linked to a carbohydrate epitope, the immune response may be to both the pan DR binding peptide and the carbohydrate epitope and optionally to other peptide sequences within the polypeptide.

### 2. Polysaccharides of the Invention

Streptococcus pneumoniae capsular polysaccharides may be used according to the methods of the invention. Over 90 serotypes of S. pneumoniae are currently known. See, e.g., Pneumococcal disease. In: Epidemiology and prevention of vaccine-preventable diseases. 6^{th} ed. Waldorf (MD): Public Health Foundation; 2000. p. 249-63; Kalin M., Thorax 53(3):159-162 (1998); Hedlund J, et al. Clin Infect Dis. 21(4):948-53 (1995). Exemplary streptococcus capsular polysaccharide antigens include, but are not limited to those from Streptococcus pneumoniae serotypes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95.

In some embodiments, the compositions of the invention comprise a mixture of capsular polysaccharides from 2, 3, 4, 5, 6, 7, 8, 9, 10, 11, 12, 13, 14, 15, 16, 17, 18, 19, 20, 21, 22, 23, 24, 25, 26, 27, 28, 29, 30 or more of the above list serotypes. In some embodiments, the compositions of the invention comprise a mixture of conjugates of a separate pan DR peptide of the invention with capsular polysaccharides from 2, 3, 4, 5, 6, 7, 8, 9, 10, 11, 12, 13, 14, 15, 16, 17, 18, 19, 20, 21, 22, 23, 24, 25, 26, 27, 28, 29, 30 or more of the following serotypes: 1, 2, 3, 4, 5, 6B, 7F, 8, 9N, 9V, 10A, 11A, 12F, 14, 15B, 17F, 18C, 19A, 19F, 20, 22F, 23F, 33F, 6A, 7A, 7B, 7C, 9A, 9L, 12A, 13, 15A, 15C, 16F, 18A, 18B, 18F, 19B, 19C, 21, 22A, 23A, 23B, 24F, 25, 27, 29, 31, 34, 35, 38, 45, or 46.

Mixtures of conjugates comprising polysaccharides of different serotypes may include, any combination of some or all of the following serotypes 1, 2, 3, 4, 5, 6B, 7F, 8, 9N, 9V, 10A, 11A, 12F, 14, 15B, 17F, 18C, 19A, 19F, 20, 22F, 23F, and 33F. In some embodiments, a combination of streptococcus capsular polysaccharide antigens includes those from Streptococcus pneumoniae serotypes 4, 6B, 9V, 14, 18C, 19F, and 23F. In some embodiments, a combination of streptococcus capsular polysaccharide antigens includes those from Streptococcus pneumoniae serotypes 1, 4, 5, 6B, 9V, 14, 18C, 19F, and 23F. In some embodiment, a combination of streptococcus capsular polysaccharide antigens includes those from Streptococcus pneumoniae serotypes 1, 3, 4, 5, 6B, 7F, 9V, 14, 18C, 19F, and 23F.

In certain embodiments, a Streptococcus pneumoniae polysaccharide/Pan DR Binding Peptide conjugate of the present invention consists of, or alternatively comprises, a Streptococcus pneumoniae polysaccharide selected from the following list of Streptococcus pneumoniae serotypes and/or serogroups: 1, 2, 3, 4, 5, 6, 6A, 6B, 7, 8, 9, 9V, 10, 11, 12, 14, 15, 15A, 16, 17, 18, 19, 19A, 19F, 20, 21, 22, 23, 23F, 24, 25, 26, 27, 28, 29, 30, 31, 32, 33, 34, 35, 35B, 36, 37, 38, 39, 40, 41, 42, 43, 44, 45, 46, 47, 48, 49, 50, 51, 52, 53, 54, 55, 56, 57, 58, 59, 61, 62, 63, 64, 65, 66, 67, 68, 69, 70, 71, 72, and 73, is conjugated using a linkage chemistry well-known in the art and/or described herein to a Pan DR Binding Peptide selected from the following list (wherein X=cyclohexlalanine):

(SEQ ID NO: 1)

aAXAAAKTAAAAa, aAXAAAATLKAAa, aAXVAAATLKAAa,
aAXIAAATLKAAa aKXVAAWTLKAAa, aKFVAAWTLKAAa,
AAXAAAKTAAAAA,

(SEQ ID NO: 2)

AAXAAAATLKAAA,

(SEQ ID NO: 3)

AAXVAAATLKAAA,

(SEQ ID NO: 4)

AAXIAAATLKAAA,

(SEQ ID NO: 5)

AKXVAAWTLKAAA,

(SEQ ID NO: 6)

AKFVAAWTLKAAA,

(SEQ ID NO: 8)

AKXVAAWTLKAAA,

(SEQ ID NO: 1)

aAFAAAKTAAAAa, aAFAAAATLKAAa, aAFVAAATLKAAa,
aAFIAAATLKAAa aKFVAAWTLKAAa, aKFVAAWTLKAAa,
AAFAAAKTAAAAA,

(SEQ ID NO: 2)

AAFAAAATLKAAA,

(SEQ ID NO: 3)

AAFVAAATLKAAA,

(SEQ ID NO: 4)

AAFIAAATLKAAA,

(SEQ ID NO: 5)

AKFVAAWTLKAAA,

(SEQ ID NO: 6)

AKFVAAWTLKAAA,

(SEQ ID NO: 9)

AKFVAAWTLKAAA,

(SEQ ID NO: _)

aKXAAAATLKAAa, aEXAAAATLKAAa, aOXAAAATLKAAa,
aQXAAAATLKAAa, aVXAAAATLKAAa, aFXAAAATLKAAa,
aAXKAAATLKAAa, aAXEAAATLKAAa, aAXQAAATLKAAa,
aAXFAAATLKAAa, aAXLAAATLKAAa, aAXAKAATLKAAa,
aAXAEAATLKAAa, aAXAQAATLKAAa, aAXAVAATLKAAa,
aAXAFAATLKAAa, aAXAAKATLKAAa, aAXAAEATLKAAa,
aAXAAQATLKAAa, aAXAAVATLKAAa, aAXAAFATLKAAa,
aAXAAAKTLKAAa, aAXAAAETLKAAa, aAXAAAQTLKAAa,
aAXAAAVTLKAAa, aAXAAAFTLKAAa, aAXAAATTLKAAa,
aAXAAAATKKAAa, aAXAAAATEKAAa, aAXAAAATQKAAa,
aAXAAAATVKAAa, aAXAAAATFKAAa, aAXAAAATIKAAa,
aAXAAAATLEAAa, aAXAAAATLQAAa. aAXAAAATLVAAa,
aAXAAAATLFAAa, aAXAAAATLRAAa, aAXAAAATLKKAa,
aAXAAAATLKEAa, aAXAAAATLKQAa, aAXAAAATLKVAa,
aAXAAAATLKFAa, aAXAAAATLKIAa, aAXAAAATLKAKa,
aAXAAAATLKAEa, aAXAAAATLKAQa, aAXAAAATLKAVa,
aAXAAAATLKAFa, aKXVKANTLKAAa, aKXVKANTLKAAa,
aKXVKAWTLKAAa, aKXVKAWTLKAAa, aKXVWANTLKAAa,
aKXVWAYTLKAAa, aKXVWAVTLKAAa, aKXVYAWTLKAAa,
aRXVRANTLKAAa, aKXVKAHTLKAAa, aKXVKAHTLKAAa,
aKXVAANTLKAAa, aKXVAANTLKAAa, aKXVAAYTLKAAa,
aKXVAAYTLKAAa, aKXVAAWTLKAAa, aKXVAAKTLKAAa,
aKXVAAHTLKAAa, aKXVAAATLKAAa, KSSaKXVMAATLKAAa,
AKXAAAATLKAAA,

(SEQ ID NO: 10)

AEXAAAATLKAAA,

(SEQ ID NO: 11)

AOXAAAATLKAAA,

(SEQ ID NO: 12)

AQXAAAATLKAAA,

(SEQ ID NO: 13)

AVXAAAATLKAAA,

(SEQ ID NO: 14)

AFXAAAATLKAAA,

(SEQ ID NO: 15)

AAXKAAATLKAAA,

(SEQ ID NO: 16)

AAXEAAATLKAAA,

(SEQ ID NO: 17)

AAXQAAATLKAAA,

(SEQ ID NO: 18)

AAXFAAATLKAAA,

(SEQ ID NO: 19)

AAXLAAATLKAAA,

(SEQ ID NO: 20)

AAXAKAATLKAAA,

(SEQ ID NO: 21)

AAXAEAATLKAAA,

(SEQ ID NO: 22)

AAXAQAATLKAAA,

(SEQ ID NO: 23)

AAXAVAATLKAAA,

(SEQ ID NO: 24)

AAXAFAATLKAAA,

(SEQ ID NO: 25)

AAXAAKATLKAAA,

(SEQ ID NO: 26)

AAXAAEATLKAAA,

(SEQ ID NO: 27)

AAXAAQATLKAAA,

(SEQ ID NO: 28)

AAXAAVATLKAAA,

(SEQ ID NO: 29)

AAXAAFATLKAAA,

(SEQ ID NO: 30)

AAXAAAKTLKAAA,

(SEQ ID NO: 31)

AAXAAAETLKAAA,

(SEQ ID NO: 32)

AAXAAAQTLKAAA,

(SEQ ID NO: 33)

AAXAAAVTLKAAA,

(SEQ ID NO: 34)

AAXAAAFTLKAAA,

(SEQ ID NO: 35)

AAXAAATTLKAAA,

(SEQ ID NO: 36)

AAXAAAATKKAAA,

(SEQ ID NO: 37)

AAXAAAATEKAAA,

(SEQ ID NO: 38)

AAXAAAATQKAAA,

(SEQ ID NO: 39)

AAXAAAATVKAAA,

(SEQ ID NO: 40)

AAXAAAATFKAAA,

(SEQ ID NO: 41)

AAXAAAATIKAAA,

(SEQ ID NO: 42)

AAXAAAATLEAAA,

(SEQ ID NO: 43)

AAXAAAATLQAAA,

(SEQ ID NO: 44)

AAXAAAATLVAAA,

(SEQ ID NO: 45)

AAXAAAATLFAAA,

(SEQ ID NO: 46)

AAXAAAATLRAAA,

(SEQ ID NO: 47)

AAXAAAATLKKAA,

(SEQ ID NO: 48)

AAXAAAATLKEAA,

(SEQ ID NO: 49)

AAXAAAATLKQAA,

(SEQ ID NO: 50)

AAXAAAATLKVAA,

(SEQ ID NO: 51)

AAXAAAATLKFAA,

(SEQ ID NO: 52)

AAXAAAATLKIAA,

(SEQ ID NO: 53)

AAXAAAATLKAKA,

(SEQ ID NO: 54)

AAXAAAATLKAEA,

(SEQ ID NO: 55)

AAXAAAATLKAQA,

(SEQ ID NO: 56)

AAXAAAATLKAVA,

(SEQ ID NO: 57)

AAXAAAATLKAFA,

(SEQ ID NO: 58)

AKXVKANTLKAAA,

(SEQ ID NO: 59)

AKXVKANTLKAAA,

(SEQ ID NO: 60)

AKXVKAWTLKAAA,

(SEQ ID NO: 61)

AKXVKAWTLKAAA,

(SEQ ID NO: 62)

AKXVWANTLKAAA,

(SEQ ID NO: 63)

AKXVWAYTLKAAA,

(SEQ ID NO: 64)

AKXVWAVTLKAAA,

(SEQ ID NO: 65)

AKXVYAWTLKAAA,

(SEQ ID NO: 66)

ARXVRANTLKAAA,

(SEQ ID NO: 67)

AKXVKAHTLKAAA,

(SEQ ID NO: 68)

AKXVKAHTLKAAA,

(SEQ ID NO: 69)

AKXVAANTLKAAA,

(SEQ ID NO: 70)

AKXVAANTLKAAA,

(SEQ ID NO: 71)

AKXVAAYTLKAAA,

(SEQ ID NO: 72)

AKXVAAYTLKAAA,

(SEQ ID NO: 73)

AKXVAAWTLKAAA,

(SEQ ID NO: 74)

AKXVAAKTLKAAA,

(SEQ ID NO: 75)

AKXVAAHTLKAAA,

(SEQ ID NO: 76)

AKXVAAATLKAAA,

(SEQ ID NO: 77)

KSSAKXVMAATLKAAA,

(SEQ ID NO: 78)

aKFAAAATLKAAa, aEFAAAATLKAAa, aOFAAAATLKAAa,
aQFAAAATLKAAa, aVFAAAATLKAAa, aFFAAAATLKAAa,
aAPKAAATLKAAa, aAFEAAATLKAAa, aAFQAAATLKAAa,
aAFFAAATLKAAa, aAFLAAATLKAAa, aAFAKAATLKAAa,
aAFAEAATLKAAa, aAFAQAATLKAAa, aAFAVAATLKAAa,
aAFAPAATLKAAa, aAFAAKATLKAAa, aAFAAEATLKAAa,
aAFAAQATLKAAa, aAFAAVATLKAAa, aAFAAFATLKAAa,
aAFAAAKTLKAAa, aAFAAAETLKAAa, aAFAAAQTLKAAa,
aAFAAAVTLKAAa, aAFAAAFTLKAAa, aAFAAATTLKAAa,
aAFAAAATKKAAa, aAFAAAATEKAAa, aAFAAAATQKAAa,
aAFAAAATVKAAa, aAFAAAATFKAAa, aAFAAAATIKAAa,
aAFAAAATLEAAa, aAFAAAATLQAAa, aAFAAAATLVAAa,
aAFAAAATLFAAa, aAFAAAATLRAAa, aAFAAAATLKKAa,
aAFAAAATLKEAa, aAFAAAATLKQAa, aAFAAAATLKVAa,
aAFAAAATLKFAa, aAFAAAATLKIAa, aAFAAAATLKAKa,
aAFAAAATLKAEa, aAFAAAATLKAQa, aAFAAAATLKAVa,
aAFAAAATLKAFa, aKFVKANThKAAa, aKFVKANTLKAAa,
aKFVKAWTLKAAa, aKFVKAWTLKAAa, aKFVWANTLKAAa,
aKFVWAYTLKAAa, aKFVWAVTLKAAa, aKFVYAWTLKAAa,
aRFVRANTLKAAa, aKFVKAHTLKAAa, aKFVKAHTLKAAa,
aKFVAANTLKAAa, aKFVAANTLKAAa, aKFVAAYTLKAAa,
aKFVAAYTLKAAa, aKFVAAWTLKAAa, aKFVAAKTLKAAa,
aKFVAAHTLKAAa, aKFVAAATLKAAa, KSSaKFVMAATLKAAa,
AKFAAAATLKAAA,

(SEQ ID NO: 79)

AEFAAAATLKAAA,

(SEQ ID NO: 80)

AOFAAAATLKAAA,

(SEQ ID NO: 81)

AQFAAAATLKAAA,

(SEQ ID NO: 82)

AVFAAAATLKAAA,

(SEQ ID NO: 83)

AFFAAAATLKAAA,

(SEQ ID NO: 84)

AAFKAAATLKAAA,

(SEQ ID NO: 85)

AAFEAAATLKAAA,

(SEQ ID NO: 86)

AAFQAAATLKAAA,

(SEQ ID NO: 87)

AAFFAAATLKAAA,

(SEQ ID NO: 88)

AAFLAAATLKAAA,

(SEQ ID NO: 89)

AAFAKAATLKAAA,

(SEQ ID NO: 90)

AAFAEAATLKAAA,

(SEQ ID NO: 91)

AAFAQAATLKAAA,

(SEQ ID NO: 92)

AAFAVAATLKAAA,

(SEQ ID NO: 93)

AAFAFAATLKAAA,

(SEQ ID NO: 94)

AAFAAKATLKAAA,

(SEQ ID NO: 95)

AAFAAEATLKAAA,

(SEQ ID NO: 96)

AAFAAQATLKAAA,

(SEQ ID NO: 97)

AAFAAVATLKAAA,

(SEQ ID NO: 98)

AAFAAFATLKAAA,

(SEQ ID NO: 99)

AAFAAAKTLKAAA,

(SEQ ID NO: 100)

AAFAAAETLKAAA,

(SEQ ID NO: 101)

AAFAAAQTLKAAA,

(SEQ ID NO: 102)

AAFAAAVTLKAAA,

(SEQ ID NO: 103)

AAFAAAFTLKAAA,

(SEQ ID NO: 104)

AAFAAATAAKAAA,

(SEQ ID NO: 105)

AAFAAAATKKAAA,

(SEQ ID NO: 106)

AAFAAAATEKAAA,

(SEQ ID NO: 107)

AAFAAAATQKAAA,

(SEQ ID NO: 108)

AAFAAAATVKAAA,

(SEQ ID NO: 109)

AAFAAAATFKAAA,

(SEQ ID NO: 110)

AAFAAAATIKAAA,

(SEQ ID NO: 111)

AAFAAAATLEAAA,

(SEQ ID NO: 112)

AAFAAAATLQAAA,

(SEQ ID NO: 113)

AAFAAAATLVAAA,

(SEQ ID NO: 114)

AAFAAAATLFAAA,

(SEQ ID NO: 115)

AAFAAAATLRAAA,

(SEQ ID NO: 116)

AAFAAAATLKKAA,

(SEQ ID NO: 117)

AAFAAAATLKEAA,

(SEQ ID NO: 118)

AAFAAAATLKQAA,

(SEQ ID NO: 119)

AAFAAAATLKVAA,

(SEQ ID NO: 120)

AAFAAAATLKFAA,

(SEQ ID NO: 121)

AAFAAAATLKIAA,

(SEQ ID NO: 122)

AAFAAAATLKAKA,

(SEQ ID NO: 123)

AAFAAAATLKAEA,

(SEQ ID NO: 124)

AAFAAAATLKAQA,

(SEQ ID NO: 125)

AAIAAAAATLKAVA,

(SEQ ID NO: 126)

AAFAAAATLKAFA,

(SEQ ID NO: 127)

AKFVKANTLKAAA,

(SEQ ID NO: 128)

AKFVKANTLKAAA,

(SEQ ID NO: 129)

AKFVKAWTLKAAA,

(SEQ ID NO: 130)

AKFVKAWTLKAAA,

(SEQ ID NO: 131)

AKFVWANTLKAAA,

(SEQ ID NO: 132)

AKFVWAYTLKAAA,

(SEQ ID NO: 133)

AKFVWAVTLKAAA,

(SEQ ID NO: 134)

AKFVYAWTLKAAA,

(SEQ ID NO: 135)

ARFVRANTLKAAA,

(SEQ ID NO: 136)

AKFVKAHTLKAAA,

(SEQ ID NO: 137)

AKFVKAHTLKAAA,

(SEQ ID NO: 138)

AKFVAANTLKAAA,

(SEQ ID NO: 139)

AKFVAANTLKAAA,

(SEQ ID NO: 140)

AKFVAAYTLKAAA,

(SEQ ID NO: 141)

AKFVAAYTLKAAA,

(SEQ ID NO: 142)

AKFVAAWTLKAAA,

(SEQ ID NO: 143)

AKFVAAKTLKAAA,

(SEQ ID NO: 144)

AKFVAAHTLKAAA,

(SEQ ID NO: 145)

AKFVAAATLKAAA,
and

(SEQ ID NO: 146)

KSSAKFVMAATLKAAA.

Accordingly, non-limiting examples of Streptococcus pneumoniae polysaccharide/Pan DR Binding Peptide conjugates of the present invention consist of, or alternatively comprise, 6B-AKFVAAWTLKAAA (SEQ ID NO:6) (wherein “6B” represents Streptococcus pneumoniae serotype 6B and “AKFVAAWTLKAAA (SEQ ID NO:6)” represents a Pan DR Binding Peptide comprising the amino acid sequence

(SEQ ID NO: 6))

AKFVAAWTLKAAA;

(SEQ ID NO: 6)

6B-aKFVAAWTLKAAa; 4-AKFVAAWTLKAAA;

(SEQ ID NO: 6)

4-aKFVAAWTLKAAa; 9V-AKFVAAWTLKAAA;

(SEQ ID NO: 6)

9V-aKFVAAWTLKAAa; 14-AKFVAAWTLKAAA;

(SEQ ID NO: 6)

14-aKFVAAWTLKAAa; 18C-AKFVAAWTLKAAA;

(SEQ ID NO: 6)

18C-aKFVAAWTLKAAa; 19F-AKFVAAWTLKAAA;

(SEQ ID NO: 6)

19F-aKFVAAWTLKAAa; 23F-AKFVAAWTLKAAA;

(SEQ ID NO: 5)

23F-aKFVAAWTLKAAa; 6B-aKXVAAWTLKAAa;
4-AKXVAAWTLKAAA;

(SEQ ID NO: 5)

4-aKXVAAWTLKAAa; 9V-AKXVAAWTLKAAA;

(SEQ ID NO: 5)

9V-aKXVAAWTLKAAa; 14-AKXVAAWTLKAAA;

(SEQ ID NO: 5)

14-aKXVAAWTLKAAa; 18C-AKXVAAWTLKAAA;

(SEQ ID NO: 5)

18C-aKXVAAWTLKAAa; 19F-AKXVAAWTLKAAA;

(SEQ ID NO: 5)

19F-aKXVAAWTLKAAa; 23F-AKXVAAWTLKAAA;
and
23F-aKFVAAWTLKAAa.

A number of different Streptococcus pneumoniae serotypes are available from the ATCC (P.O. Box 1549, Manassas, Va. 20108), including those listed in Table 1.

TABLE 1
List of representative serotype bacterial cultures available from ATCC

| Serotype | ATCC No. or Nos. |
|---|---|
| 1 | 6301, 9163 |
| 2 | 6302 |
| 3 | 6303 |
| 4 | 6304, BAA-334 |
| 5 | 6305, BAA-341 |
| 6 | 6306 |
| 6A | BAA-659 |
| 6B | BAA-342, BAA-612, BAA-658, 51937, |
| | 700670, 700675, 700903 |
| 7 | 6307 |
| 8 | 6308 |
| 9 | 6309 |
| 9V | 700671 |
| 10 | 6310 |
| 11 | 6311 |
| 12 | 6312 |
| 14 | 6314, BAA-340, 51936, 700672, 700676, |
| | 700678, 700902 |
| 15 | 6315 |
| 15A | BAA-661 |
| 16 | 6316 |
| 17 | 6317 |
| 18 | 6318 |
| 19 | 6319 |
| 19A | 700673, 700674, 700678, 700904, BAA-475 |
| 19F | 49619, 700905, BAA-657 |
| 20 | 6320 |
| 21 | 6321 |
| 22 | 6322 |
| 23 | 6323 |
| 23F | 51938, 700669, BAA-943 |
| 24 | 6324 |
| 25 | 6325 |
| 26 | 6326 |
| 27 | 6327 |
| 28 | 6328 |
| 29 | 6329 |
| 30 | 6330 |
| 31 | 6331 |
| 32 | 6332 |
| 33 | 8333 |
| 34 | 8334 |
| 35 | 8335 |
| 35B | BAA-660 |
| 36 | 8336 |
| 37 | 8337 |
| 38 | 8338 |
| 39 | 8339 |
| 40 | 8340 |
| 41 | 10341 |
| 42 | 10342 |
| 43 | 10343 |
| 44 | 10344 |
| 45 | 10345 |
| 46 | 10346 |
| 47 | 10347 |
| 48 | 10348 |
| 49 | 10349 |
| 50 | 10350 |
| 51 | 10351 |
| 52 | 10352 |
| 53 | 10353 |
| 54 | 10354 |
| 55 | 10355 |
| 56 | 10356 |
| 57 | 10357 |
| 58 | 10358 |
| 59 | 10359 |
| 61 | 10361 |
| 62 | 10362 |
| 63 | 10363 |
| 64 | 10364 |
| 65 | 10365 |
| 66 | 10336 |
| 67 | 10367 |
| 68 | 10368 |
| 69 | 10369 |
| 70 | 10370 |
| 71 | 10371 |
| 72 | 10372 |
| 73 | 10373 |

Purified capsular polysaccharides from at least serotypes 1, 2, 3, 4, 5, 8, 9N, 12F, 14, 17F, 19F, 20, 22F, 23F, 25, 6B, 10A, 11A, 7F, 15B, 18C, 19A, 9V, and 33F are available from the ATCC.

The chemical structures of a number of serotype capsular polysaccharides have been determined. For example, van Selm, et al, Infect. Immun. 71(11):6192-6198 (2003) provides the structures of the serotype 14, 15B and 15C capsular polysaccharides as follows:

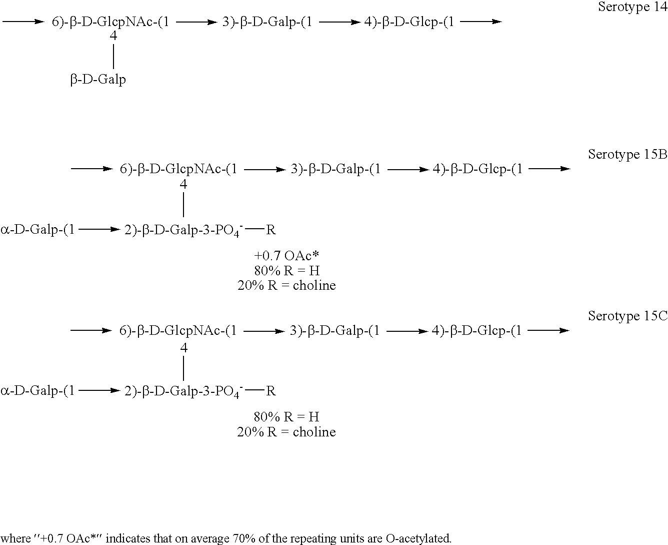

Jiang et al., Infect. Immun. 69(3):1244-1255 (2001) provides the structures of serotype 4, 6B, 8, or 18C capsular polysaccharides as follows:

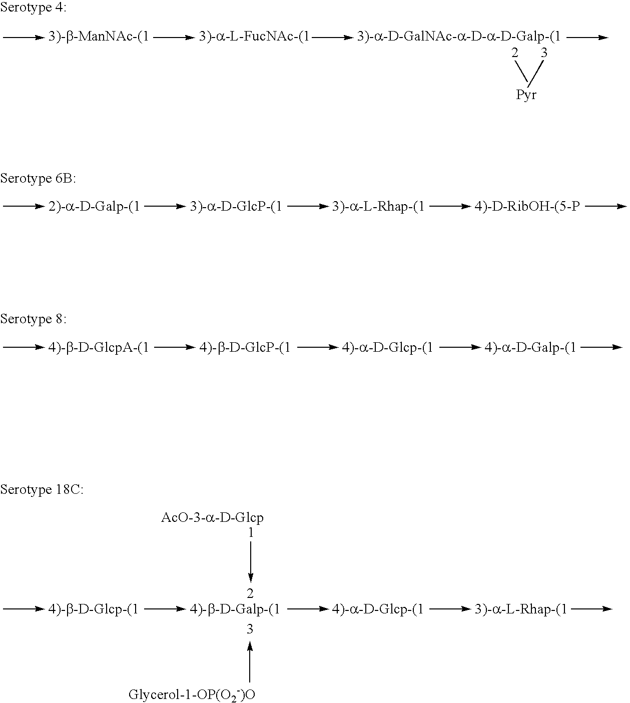

Morona et al., J. Bacterial 181(17):5355-5364 (1999) provides the structures of serotype 19F, 19A, 19B and 19C capsular polysaccharides as follows:

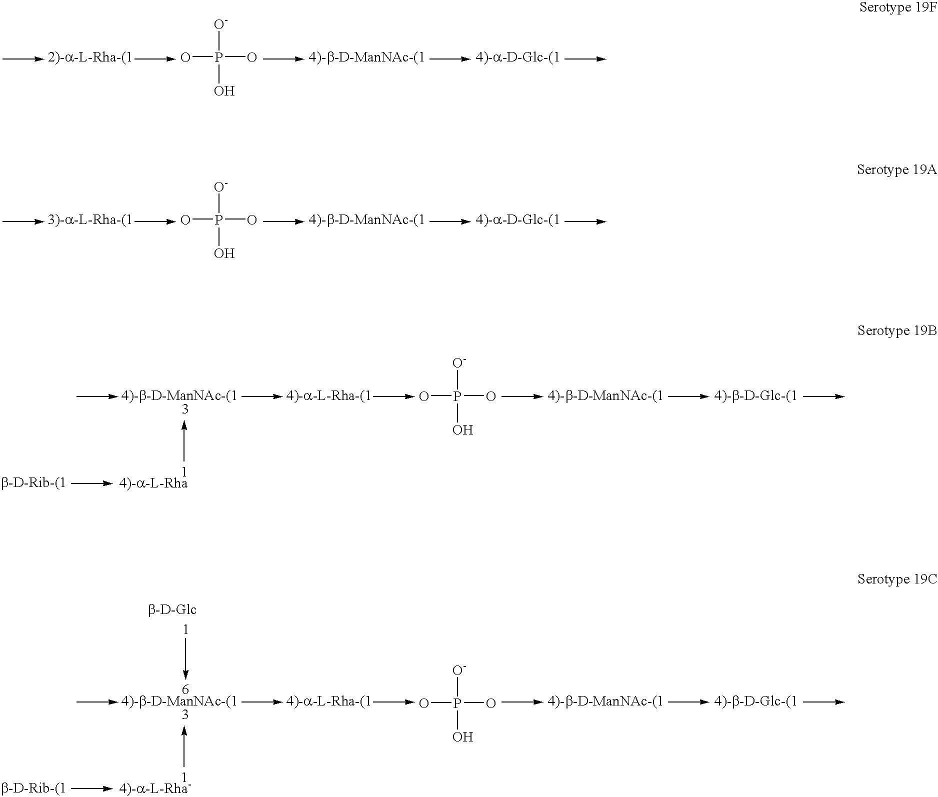

Karlsson et al., Eur. J. Biochem. 255:296-302 (1998) provides the structures of serotype 32A and 32F capsular polysaccharides as follows:

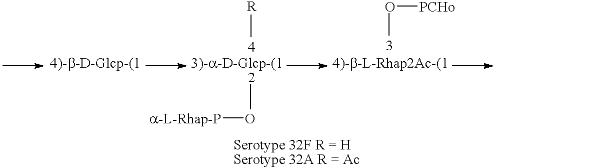

The carbohydrates used in the present invention can be prepared according to standard procedures known to those of skill in the art. Streptococcus polysaccharides may be prepared from their respective bacterial strains, by any method known in the art. In an exemplary method, Streptococcus polysaccharides are purified from bacterial strains by contacting the bacterial components with a base reagent to obtain a mixture wherein the pH of the mixture is between about, e.g., 9 and 14, (e.g., wherein pH=9, pH=9.5, pH=10, pH=10.5, pH=11, pH=11.5, pH=12, pH=12.5, pH=13, pH=13.5 or pH=14), separating the capsular polysaccharides from the cellular components, and recovering the capsular polysaccharides substantially free of the other cellular components. See, e.g., U.S. Pat. No. 6,248,570. See also, Wessels, M. R., et al. Infect. Immun. 57:1089-1094 (1989); Wessels, M. R., et al. J. Clin. Invest. 86:1428-1433 (1990).

Alternatively, the polysaccharides of the invention may be prepared from suitable monomeric sugars through the formation of glycosidic linkages or isolated from natural sources and modified as appropriate. For example, a β-glycosyl bond can be formed between one sugar bearing a 1-halo substituent and a second, suitably protected sugar having at least one unprotected hydroxyl group. Such transformation are typically carried out in the presence of silver carbonate (Ag₂CO₃) or silver triflate.

Alternatively, the glycosidic linkages can be formed by enzymatic means, using methods described in International Patent Application Publication No. WO 96/32492. Briefly, glycosyltransferases such as sialyltransferase can be utilized for the construction of specific glycosidic linkages.

A number of sialyltransferases are known to those of skill in the art. This enzyme transfers sialic acid (NeuAc) to a Gal with the formation of an α-linkage between the two saccharides. Bonding (linkage) between the saccharides is between the 2-position of NeuAc and the 3-position of Gal.

An exemplary α(2,3)sialyltransferase (EC 2.4.99.6) often referred to as sialyltransferase, transfers sialic acid to the non-reducing terminal Gal of a Galβ1→3Glc disaccharide or glycoside. See, e.g., Van den Eijnden, et al., J. Biol. Chem., 256:3159 (1981), Weinstein, et al., J. Biol. Chem., 257:13845 (1982) and Wen, et al., J. Biol. Chem., 267:21011 (1992). Another exemplary α-2,3-sialyltransferase (EC 2.4.99.4) transfers sialic acid to the non-reducing terminal Gal of the disaccharide or glycoside. See, e.g., Rearick, et al., J. Biol. Chem., 254:4444 (1979) and Gillespie, et al., J. Biol. Chem., 267:21004 (1992). Further exemplary enzymes include Gal-β-1,4-GlcNAc α-2,6 sialyltransferase. See, e.g., Kurosawa, et al. Eur. J. Biochem. 219:375-381 (1994)).

One of skill in the art will understand that other glycosyltransferases can be substituted into similar transferase cycles as have been described in detail for the sialyltransferase. For instance, the glycosyltransferase can also be, for instance, glucosyltransferases, e.g., Alg8 (Stagljov, et al., Proc. Natl. Acad. Sci. USA 91:5977 (1994)) or Alg5 (Heesen, et al. Eur. J. Biochem. 224:71 (1994)). Suitable N-acetylgalactosaminyltransferases include α(1,3)N-acetylgalactosaminyltransferase, β(1,4)N-acetylgalactosaminyltransferases (Nagata, et al. J. Biol. Chem. 267:12082-12089 (1992) and Smith, et al. J. Biol. Chem. 269:15162 (1994)) and polypeptide N-acetylgalactosaminyltransferase (Homa, et. al. J. Biol Chem. 268:12609 (1993)). Suitable N-acetylglucosaminyltransferases include GnTI (2.4.1.101, Hull, et al., BBRC 176:608 (1991)), GnTII, and GnTIII (Ihara, et al., J. Biolchem. 113:692 (1993)), GnTV (Shoreiban, et al., J. Biol. Chem. 268:15381 (1993)), O-linked N-acetylglucosaminyltransferase (Bierhuizen, et al., Proc. Natl. Acad. Sci. USA 89:9326 (1992)), and hyaluronan synthase. Suitable mannosyltransferases include α(1,2)mannosyltransferase, α(1,3)mannosyltransferase, β(1,4)mannosyltransferase, Dol-P-Man synthase, OCh1, and Pmtl.

Other suitable glycosyltransferase cycles are described in Ichikawa, et al, J. Am. Chem. Soc. 114:9283 (1992), Wong, et al., J. Org. Chem. 57:4343 (1992), DeLuca, et al., J. Am. Chem. Soc. 117:5869-5870 (1995), and Ichikawa, et al., in Carbohydrates and Carbohydrate Polymers, Yaltami, ed. (ATL Press, 1993).

For the above glycosyltransferase cycles, the concentrations or amounts of the various reactants used in the processes depend upon numerous factors including reaction conditions such as temperature and pH, and the choice and amount of acceptor saccharides to be glycosylated. Because the glycosylation process permits regeneration of activating nucleotides, activated donor sugars and scavenging of produced pyrophosphate in the presence of catalytic amounts of the enzymes, the process is limited by the concentrations or amounts of the stoichiometric substrates discussed before. The upper limit for the concentrations of reactants that can be used in accordance with the method of the present invention is determined by the solubility of such reactants.

CTL peptides comprising additional polypeptides (e.g., from S. pneumoniae-derived polypeptides) may also be administered with the polypeptide/polysaccharide conjugates of the invention. CTL peptides comprising appropriate epitopes may be synthesized and then tested for their ability to bind to MHC Class I molecules in assays using, for example, purified class I molecules and iodinated peptides and/or cells expressing empty class I molecules by, for instance, immunofluorescent staining and flow microfluorimetry, peptide-dependent class I assembly assays, and inhibition of CTL recognition by peptide competition. Those peptides that bind to the class I molecule may be further evaluated for their ability to serve as targets for CTLs derived from infected or immunized individuals, as well as for their capacity to induce primary in vitro or in vivo CTL responses that can give rise to CTL populations capable of reacting with virally infected target cells or tumor cells as potential therapeutic agents.

In certain embodiments, antibody-inducing peptides, i.e., peptides comprising an antibody epitope, may be administered with the polypeptide/polysaccharide conjugates of the invention.

The one or more CTL and/or antibody-inducing peptides may be administered with one or more pan DR peptides in a mixture which may or may not involve noncovalent associations between the peptides. For instance, one or more of the peptides may be lipidated. Alternatively, the peptides may be covalently linked (e.g., in a recombinant fusion).

To facilitate the association of the antigenic determinant with the pap DR binding peptide, additional amino acids can be added to the termini of the peptides. The additional residues can also be used for coupling to a carrier, support or larger peptide, for reasons discussed herein, or for modifying the physical or chemical properties of the peptide or oligopeptide, or the like. Amino acids such as tyrosine, cysteine, lysine, glutamic or aspartic acid, or the like, can be introduced at the C- or N-terminus of the peptide or oligopeptide. In addition, the peptide or oligopeptide sequences can differ from the natural sequence by being modified by terminal-NH₂ acylation, e.g., by alkanoyl (C₁-C₂₀) or thioglycolyl acetylation, terminal-carboxy amidation, e.g., ammonia, methylamine, etc. In some instances these modifications may provide sites for linking to a support or other molecule.

As with the pan DR binding peptides, it will be understood that the antigenic determinants may be modified to provide other desired attributes, e.g., improved pharmacological characteristics, while increasing or at least retaining substantially all of the biological activity of the unmodified peptide. For instance, the peptides can be modified by extending, decreasing or substituting in the peptides amino acid sequences by the addition or deletion of amino acids on either the amino terminal or carboxy terminal end, or both, of peptides derived from the sequences disclosed herein. Usually, the portion of the sequence which is intended to substantially mimic a CTL- or antibody-stimulating epitope will not differ by more than about 20% from the sequence of the target antigenic protein, except where additional amino acids may be added at either terminus for the purpose of modifying the physical or chemical properties of the peptide for ease of linking or coupling, and the like. In situations where regions of the peptide sequences are found to be polymorphic among viral subtypes, it may be desirable to vary one or more particular amino acids to more effectively mimic differing cytotoxic T-lymphocyte epitopes of different viral strains or serotypes.

The peptides of the invention can be combined via linkage to form polymers (multimers), or can be formulated in a composition without linkage, as an admixture. Where a peptide is linked to an identical peptide, thereby forming a homopolymer, a plurality of repeating epitopic units are presented. For example, multiple antigen peptide (MAP) technology can be used to construct polymers containing both CTL and/or antibody peptides and pan DR binding peptides. When the peptides differ, e.g., a cocktail representing different viral subtypes, different epitopes within a subtype, different HLA restriction specificities, or peptides which contain T helper epitopes, heteropolymers with repeating units may be provided. In addition to covalent linkages, noncovalent linkages capable of forming intermolecular and intrastructural bonds are also contemplated.

### 3. Preparation of Conjugates

Desired polysaccharides may be conjugated to the polypeptides comprising pan DR peptides sequences by numerous methods. Polysaccharides may be linked to the polypeptides of the invention by enzymatic or chemical reactions. For example, a wide range of linking strategies are described in Hermanson, BIOCONJUGATE TECHNIQUES (Academic Press, 1996); Lockhart, “Conjugate Vaccines,” Expert Rev. Vaccines 2(5):633-648 (2003). Ionic interactions are possible through the term in or through the ε-amino group of lysine. Hydrogen bonding between the side groups of the residues and the antigenic determinants are also possible.

In some embodiments, polysaccharide/pan DR binding peptide conjugates are linked by a spacer molecule or linker. Alternatively, the polysaccharide may be attached directly to the pan DR binding peptide without a linker.

The spacer or linker may be comprised of neutral molecules, such as, aliphatic carbon chains, amino acids or amino acid mimetics, which are substantially uncharged under physiological conditions and may have linear or branched side chains. A number of compositions and methods for linking various biomolecules are known to those of skill in the art. The particular method by which a pan DR binding peptide is covalently linked, for instance, to a carbohydrate epitope may vary. Methods suitable for linking pan DR binding peptides to carbohydrate antigens are disclosed for instance in WO 93/21948.

A number of linkers are well known and are either commercially available or are described in the scientific literature. Exemplary linkers include, e.g., homo- and hetero-bifunctional linkers. The linking molecules used in the present invention may be optionally of sufficient length to permit the two portions of the molecule to interact independently and freely with molecules exposed to them. In the case of carbohydrate epitopes, the linking molecules are typically 1-50 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50) atoms long. For example, the linking molecules may be aryl acetylene, ethylene glycol oligomers containing 2-14 (e.g., 2, 3, 4, 5, 6, 7, 8, 9, 10, 11, 12, 13 or 14) monomer units, diamines, diacids, amino acids, or combinations thereof. Other suitable linkers include lipid molecules such as ceramide and amino acid residues to which a different carbohydrate moiety is linked through the amino acid side chain.

The particular linking molecule used may be selected based upon its chemical/physical properties. The linking molecule has an appropriate functional group at each end, one group appropriate for attachment to the reactive sites on the carbohydrate portion and the other group appropriate for attachment to the amino acid/peptide portion. For example, groups appropriate for attachment to the carbohydrate portion are carboxylic acid, ester, isocyanate, alkyl halide, acyl halide and isothiocyanate. Similar groups would be useful for attachment to the amino acid portion. Appropriate selection of the functional group will depend on the nature of the reactive portion of the amino acid or peptide.

Indirect binding can be achieved using a variety of linkers that are commercially available. The reactive ends can be any of a variety of functionalities including, but not limited to: amino reacting ends such as N-hydroxysuccinimide (NHS) active esters, imidoesters, aldehydes, epoxides, sulfonyl halides, isocyanate, isothiocyanate, and nitroaryl halides; and thiol reacting ends such as pyridyl disulfides, maleimides, thiophthalimides, and active halogens. Crosslinking agents and other bioconjugates are discussed in detail in Hermanson, G. T., BIOCONJUGATE TECHNIQUES (Academic Press, Inc. 1996) and the PIERCE (Pierce/Endogen) 2001-2002 Catalog. In some embodiments, the polysaccharide is converted into an activated hydrazide (e.g., with adipic acid dihydrizde in NaHCO₃). The activated polysaccharide hydrazide may be then conjugated to a polypeptide comprising the pan-DR binding peptide sequence via the polypeptide carboxyl group(s) by carbodiimide-mediated condensation.

In addition, a bifunctional linker having one functional group reactive with a group on a particular ligand, and another group reactive with a nucleic acid binding molecule, can be used to form the desired conjugate. Heterobifunctional crosslinking reagents have two different reactive ends, e.g., an amino-reactive end and a thiol-reactive end, while homobifunctional reagents have two similar reactive ends, e.g., bismaleimidohexane (BMH) which permits the cross-linking of sulfhydryl-containing compounds. The spacer can be aliphatic or aromatic. Heterobifunctional reagents include commercially available active halogen-NHS active esters coupling agents such as N-succinimidyl bromoacetate and N-succinimidyl(4-iodoacetyl)aminobenzoate (SIAB) and the sulfosuccinimidyl derivatives such as sulfosuccinimidyl(4-iodoacetyl)aminobenzoate (sulfo-SIAB) (Pierce). Another group of coupling agents is the heterobifunctional and thiol cleavable agents such as N-succinimidyl 3-(2-pyridyidithio)propionate (SPDP) (Pierce). Other bifunctional linkers include, e.g., ABH (p-Azidobenzoyl Hydrazide); BMPH (N-[b-Maleimidoproprionic acid]hydrazide TFA), and KMUH (N-[k-Maleimidoundecanoic acid]hydrazide, M2C2H (4-[N-Maleimidomethyl]cyclohexanne-1-careboxylhydrazide HCl ½ dioxane), and MPBH (4-[4-N-Maleimidophenyl]butyric acid hydrazide HCl).

Heterobifunctional linkers, such as maleimide-hydroxysuccinimide ester, can also be used as selective linkages (see, e.g., U.S. Pat. No. 5,851,527). Reaction of maleimide-hydroxysuccinimide ester with a polypeptide target will derivatize amine groups on the protein, and the derivative can then be reacted with, e.g., a target with free sulfhydryl groups. Many other procedures and linker molecules for attachment of various compounds to proteins are known. See, for example, European Patent Application No. 188,256; U.S. Pat. Nos. 4,671,958; 4,659,839; 4,414,148; 4,699,784; 4,680,338; 4,569,789; 5,856,571; 5,824,805; 5,470,997; 5,470,843; 5,470,932; 5,843,937 and 4,589,071; and Borlinghaus et al. Cancer Res. 47:4071-4075 (1987).

Examples of commercially available homobifunctional cross-linking reagents include, but are not limited to, the imidoesters such as dimethyl adipimidate dihydrochloride (DMA); dimethyl pimelimidate dihydrochloride (DMP); and dimethyl suberimidate dihydrochloride (DMS).

In one group of embodiments, alkyl or alkylene groups will be useful as linking groups and will have 1 to 20 carbon atoms (e.g., 1, 2, 3, 4, 5, 6, 7, 8, 9, 10, 11, 12, 13, 14, 15, 16, 17, 18, 19 or 20). For instance, linkers comprising polyethylene glycol and related structures can be used. The term “polyethylene glycol” is used to refer to those molecules which have repeating units of ethylene glycol, for example, hexaethylene glycol (HO—(CH₂CH₂O)₅—CH₂CH₂OH). When the term “polyethylene glycol” is used to refer to linking groups, it would be understood by one of skill in the art that other polyethers or polyols could be used as well (i.e, polypropylene glycol or mixtures of ethylene and propylene glycols).

In another group of embodiments, the alkyl or alkylene linking groups will be perfluorinated, rendering them less susceptible to biological degradation. See, U.S. Pat. No. 5,055,562. Exemplary linking groups will include aminocaproic acid, 4-hydroxy butyric acid, 4-mercapto butyric acid, 3-amino-1-propanol, ethanolamine, perfluoroethanolamine, and perfluorohydroxybutyric acid. In one group of embodiments, the two portions are linked via a polyethylene glycol moiety.

In the case of linkers between pan DR binding peptides and other peptides (e.g., a pan DR binding peptide and a CTL inducing peptide), the spacers may be, e.g., selected from Ala, Gly, or other neutral spacers of nonpolar amino acids or neutral polar amino acids. In some embodiments herein the neutral spacer is Ala. It will be understood that the optionally present spacer need not be comprised of the same residues and thus may be a hetero- or homo-oligomer. Exemplary spacers may be homo-oligomers of Ala. When present, the spacer will usually be at least one or two residues, e.g., three to six residues (i.e., 3, 4, 5 or 6). In other embodiments the pan DR binding peptide is conjugated to the CTL or antibody-inducing peptide, preferably with the pan DR binding peptide positioned at the amino terminus. The peptides may be joined by a neutral linker, such as Ala-Ala-Ala or the like, and preferably further contain a lipid residue such as palmitic acid or the like which is attached to alpha and epsilon amino groups of a Lys residue ((PAM)₂Lys), which is attached to the amino terminus of the peptide conjugate, typically via Ser-Ser linkage or the like.

The CTL or antibody-inducing peptide may be linked to the pan DR binding peptide either directly or via a spacer either at the amino or carboxy terminus of the CTL peptide. The amino terminus of either the CTL or antibody-inducing peptide or the pan DR binding peptide may be acylated. In addition, the CTL peptide/pan DR binding conjugate may be linked to certain alkanoyl (C₁-C₂₀) lipids via one or more linking residues such as Gly, Gly-Gly, Ser, Ser-Ser as described below. Other useful lipid moieties include cholesterol, fatty acids, and the like.

In some embodiments it may be desirable to include in the pharmaceutical compositions of the invention at least one component which assists in priming CTL. Lipids have been identified as agents capable of assisting the priming CTL in vivo against viral antigens. For example, steroids such as cholesterol, fatty acids such as palmitic acid residues can be attached to the sulfhydryl group of a cysteine residue, the alpha and epsilon amino groups of a Lys residue and then linked, e.g., via one or more linking residues such as Gly, Gly-Gly-, Ser, Ser-Ser, or the like, to an immunogenic peptide, such as a pan DR binding peptide. Alternatively, in place of fatty acids, long chain alkyl groups can be linked through an ether linkage to the final amino acid (e.g., a cysteine residue).

The lipidated peptide can be injected, either directly in a micellar form, incorporated into a liposome or emulsified in an adjuvant, e.g., incomplete Freund's adjuvant. In some embodiments, a particularly effective immunogen comprises palmitic acid attached to alpha and epsilon amino groups of Lys, which is attached via linkage, e.g., Ser-Ser, to the amino terminus of the immunogenic peptide.

As another example of lipid priming of CTL responses, E. coli lipoproteins, such as tripalmitoyl-S-glycerylcysteinlyseryl-serine (P₃CSS) can be used to prime virus specific CTL when covalently attached to an appropriate peptide. See, Deres, et al., Nature 342:561-564 (1989). Peptides of the invention can be coupled to P₃CSS, for example, and the lipopeptide administered to an individual to specifically prime a CTL response to the target antigen. Further, as the induction of neutralizing antibodies can also be primed with P₃CSS conjugated to a peptide which displays an appropriate epitope, the two compositions can be combined to more effectively elicit both humoral and cell-mediated responses to infection.

In the case of pan DR binding peptides conjugated to carbohydrate epitopes, the lipid moieties may be linked to the opposite terminus of the carbohydrate (e.g., carbohydrate linked to the C-terminus and lipid linked to the N-terminus). Alternatively, both the lipid and the carbohydrate moieties may be linked to the same end of the peptide. For instance, the two moieties may be linked to the same linker on the N-terminus.

### 4. Pharmaceutical Compositions

The compounds of the present invention, and pharmaceutical and vaccine compositions thereof, can be administered to mammals, particularly humans, for prophylactic and/or therapeutic purposes. The present invention can be used to elicit and/or enhance immune responses against polysaccharide or peptide immunogens. For instance, Streptococcus polysaccharide/pan DR binding peptide mixtures may be used to treat and/or prevent bacterial infection. In some embodiments, Streptococcus polysaccharide/pan DR binding peptide conjugates of the invention are used to treat and/or prevent middle ear infections, pneumonia, and meningitis associated with infection of humans or other mammals with Streptococcus pneumoniae.

The present invention is directed to vaccines which contain as an active ingredient an immunogenically effective amount of a composition comprising at least one conjugate of the present invention as described herein. The vaccines can also contain a physiologically tolerable (acceptable) diluent such as water, phosphate buffered saline, or saline, and further typically include an adjuvant. Adjuvants such as incomplete Freund's adjuvant, alhydrogel Al(OH)₃, aluminum phosphate, aluminum hydroxide, or alum are materials well known in the art. And, as mentioned above, immune responses can be primed by conjugating compositions of the present invention to lipids, such as P₃CSS. Upon immunization with a composition as described herein, via injection, aerosol, oral, transdermal or other route, the immune system of the host responds by producing an enhanced immune response, humoral and/or cellular.

Vaccine compositions of the invention may be administered to a patient susceptible to or otherwise at risk of disease, including, e.g., children under 2 or the elderly (over 65), to elicit and/or enhance an immune response against an antigenic determinant. Such an amount is defined to be an “immunogenically effective dose,” either for therapeutic or prophylactic use. In this use, the precise amounts again depend on the patient's state of health and weight, the mode of administration, the nature of the formulation, etc., but generally range from about 1.0 μg to about 5000 μg per 70 kilogram patient, more commonly from about 10 μg to about 500 μg per 70 kg of body weight.

In some instances it may be desirable to combine the compositions of the present invention with vaccines which induce neutralizing antibody responses to other infections and cancers of interest.

Many different techniques exist for the timing of the immunizations when a multiple administration regimen is utilized. It is possible to use the compositions of the invention more than once to increase the levels and diversities of expression of the immunoglobulin repertoire expressed by the immunized animal. Typically, if multiple immunizations are given, they will be given one to two months apart.

In therapeutic applications, the present invention is administered to an individual already suffering from cancer, or infected with the microorganism of interest. Those in the incubation phase or the acute phase of the disease may be treated with the present invention separately or in conjunction with other treatments, as appropriate.

In therapeutic applications, a composition of the present invention is administered to a patient in an amount sufficient to elicit an effective CTL response or humoral response to the microorganism or tumor antigen and to cure, or at least partially arrest, symptoms and/or complications. An amount adequate to accomplish this is defined as “therapeutically effective dose.” Amounts effective for this use will depend in part on the peptide composition, the manner of administration, the stage and severity of the disease being treated, the weight and general state of health of the patient, and the judgment of the prescribing physician.

Therapeutically effective amounts of the compositions of the present invention generally range for the initial immunization that is for therapeutic or prophylactic administration, from about 1.0 μg to about 10,000 μg of peptide for a 70 kg patient, usually from about 100 to about 8060 μg, and preferably between about 200 and about 6000 μg.

These doses are followed by boosting dosages of from about 1.0 μg to about 1000 μg of peptide pursuant to a boosting regimen over weeks to months depending upon the patient's response and condition by measuring specific immune responses.

It must be kept in mind that the compositions of the present invention may generally be employed in serious disease states, that is, life-threatening or potentially life threatening situations. In such cases, in view of the minimization of extraneous substances and the relative nontoxic nature of the conjugates, it is possible and may be felt desirable by the treating physician to administer substantial excesses of these compositions. Further, the present invention can be used prophylactically to prevent and/or ameliorate bacterial infections, viral infections, fungal infections, parasitic infections and cancer. Effective amounts are as described above. Additionally, one of ordinary skill in the vaccine arts would also know how to adjust or modify prophylactic treatments, as appropriate, for example by boosting and adjusting dosages and dosing regimes.

Therapeutic administration may begin at the first sign of disease or the detection or surgical removal of tumors or shortly after diagnosis in the case of acute infection. This is followed by boosting doses until symptoms are substantially abated and for a period thereafter. In chronic infection, initial high doses followed by boosting doses may be required.

Treatment of an infected individual with the compositions of the invention may hasten resolution of the infection in acutely infected individuals. For those individuals susceptible (or predisposed) to developing chronic infection the compositions are particularly useful in methods for preventing the evolution from acute to chronic infection. Where the susceptible individuals are identified prior to or during infection, for instance, as described herein, the composition can be targeted to them, minimizing need for administration to a larger population.

The present invention can also be used for the treatment of chronic infection and to stimulate the immune system to eliminate virus-infected cells in individuals with latent infections. It is important to provide an amount of compositions of the present invention in a formulation and mode of administration sufficient to effectively elicit and/or enhance an immune response. Thus, for treatment of chronic infection, a representative dose is in the range of about 1.0 μg to about 5000 μg, preferably about 5 μg to 1000 μg for a 70 kg patient per dose. Immunizing doses followed by boosting doses at established intervals, e.g., from one to four weeks, may be required, possibly for a prolonged period of time to effectively immunize an individual. In the case of chronic infection, administration should continue until at least clinical symptoms or laboratory tests indicate that the viral infection has been eliminated or substantially abated and for a period thereafter.

The pharmaceutical compositions for therapeutic or prophylactic treatment are intended for parenteral, topical, oral or local administration. Typically, the pharmaceutical compositions are administered parenterally, e.g., intravenously, subcutaneously, intradermally, or intramuscularly. Because of the ease of administration, the vaccine compositions of the invention are particularly suitable for oral administration. Thus, the invention provides compositions for parenteral administration which comprise a solution of the peptides or conjugates dissolved or suspended in an acceptable carrier, preferably an aqueous carrier. A variety of aqueous carriers may be used, e.g., water, buffered water, 0.9% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as buffering agents, tonicity adjusting agents, wetting agents and the like, for example, sodium acetate, sodium lactate, sodium chloride, potassium chloride, calcium chloride, sorbitan monolaurate, triethanolamine oleate, etc.

The concentration of compositions of the present invention in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

The present invention may also be administered via liposomes, which serve to target the conjugates to a particular tissue, such as lymphoid tissue, or targeted selectively to infected cells, as well as increase the half-life of the peptide composition. Liposomes include emulsions, foams, micelles, insoluble monolayers, liquid crystals, phospholipid dispersions, lamellar layers and the like. In these preparations the composition to be delivered is incorporated as part of a liposome, alone or in conjunction with a molecule which binds to, for example, a receptor prevalent among lymphoid cells. These molecules would include monoclonal antibodies which bind to the CD45 antigen, or with other therapeutic or immunogenic compositions. Thus, liposomes filled with a desired composition of the present invention can be directed to the site of lymphoid cells, where the liposomes then deliver the selected therapeutic/immunogenic peptide compositions. Liposomes for use in the invention are formed from standard vesicle-forming lipids, which generally include neutral and negatively charged phospholipids and a sterol, such as cholesterol. The selection of lipids is generally guided by consideration of liposome size, acid lability and stability of the liposomes in the blood stream. A variety of methods are available for preparing liposomes, as described in, e.g., Szoka, et al., Ann. Rev. Biophys. Bioeng. 9:467 (1980), U.S. Pat. Nos. 4,235,871, 4,501,728, 4,837,028, and 5,019,369, incorporated herein by reference.

For targeting to the immune cells, a ligand to be incorporated into the liposome can include antibodies or fragments thereof specific for cell surface determinants of the desired immune system cells. A liposome suspension containing a composition of the present invention may be administered intravenously, locally, topically, etc. in a dose which varies according to, inter alia, the manner of administration, the composition being delivered, and the stage of the disease being treated.

Alternatively, DNA or RNA encoding both one or more pan DR binding peptides and a polypeptide containing one or more CTL epitopes or antibody inducing epitopes may be introduced into individuals to obtain an immune response to the polypeptides which the nucleic acid encodes. Wolff, et. al., Science 247: 1465-1468 (1990) describes the expression of polypeptides which nucleic acids encode.

For solid compositions, conventional nontoxic solid carriers may be used. These may include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more conjugates of the invention, and more preferably at a concentration of 25-75%.

For aerosol administration, the compositions of the present invention are preferably supplied in finely divided form along with a surfactant and propellant. Typical percentages of the composition are 0.01-20% by weight, preferably 1-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1-20% by weight of the composition, preferably 0.25-5%. The balance of the composition is ordinarily propellant. A carrier can also be included, if desired, as with lecithin for intranasal delivery.

The compositions of the present invention may also find use as diagnostic reagents. For example, a composition of the invention may be used to determine the susceptibility of a particular individual to a treatment regimen which employs the antigenic determinants, and thus may be helpful in modifying an existing treatment protocol or in determining a prognosis for an affected individual. In addition, the compositions of the present invention may also be used to predict which individuals will be at substantial risk for developing chronic infection.

### Example

The following example is offered by way of illustration and not by way of limitation.

Experimental carbohydrate-conjugate vaccines composed of the 13 amino acid universal Pan HLA-DR epitope and Streptococcus pneumoniae capsular polysaccharides from serotypes 14, 6B and 9V were produced. Simple carbodiimide-mediated condensation chemistry was used to conjugate the pan DR binding synthetic peptide to the three chemically different capsular polysaccharides in a 1:1 molar ratio. The immunogenicity of the pan DR binding peptide component of the conjugate vaccines was confirmed by the induction of pan DR binding peptide-specific CD4+ helper T cell (HTL) responses following immunization of C57BL/6 mice. High titer antibody responses specific for polysaccharides of S. pneumoniae serotypes 14, 6B and 9V were induced using Complete Freund's Adjuvant and alhydrogel Al(OH)₃ formulations. The HTL, or carrier, effect of the pan DR binding synthetic peptide was only evident using the pan DR binding peptide-polysaccharide conjugates; simple mixtures of the pan DR binding peptide and polysaccharides were essentially nonimmunogenic. The functional or potential protective value of the polysaccharide-specific antibodies was measured as a function of opsonophagocytic activity for the 6B serotype. High titers of opsonophagocytic activity were measured in sera from mice immunized with formulations containing both adjuvants. These data demonstrate that the pan DR binding synthetic peptide can induce the HTL responses needed to support the development of antibodies specific for bacterial carbohydrates used in conjugate vaccines. Details of these experiments are provided below.

### Methods and Materials

Pan DR Binding Peptide Synthesis and Conjugation of S. pneumoniae Polysaccharides

A pan DR binding peptide was designed to bind with moderate-high affinity to the most common HLA-DR allelic products with charged or bulky amino acids of the epitope as T cell receptor (TCR) contact points (Alexander J, et al. Immunity 1(9):751-61 (1994); Alexander J, et al., J. Immunol 164(3):1625-33 (2000)). The pan DR binding peptide binds to murine I-A^{b} molecules and is immunogenic in C57BL/6 mice; making is possible to evaluate conjugate vaccine immunogenicity in vivo in a well characterized animal model species. The pan DR binding synthetic peptide, with the sequence of AKXVAAWTLKAAA (SEQ ID NO:5) where X=cyclohexylalanine, was synthesized using standard F-moc solid phase synthesis methods (Ruppert J, et al. Cell 74(5):929-37 (1993)). The pan DR binding peptide was purified to >95% homogeneity by reverse-phase high pressure liquid chromatography and characterized by mass spectrometry.

Capsular polysaccharide preparations from serotypes 6B, 9V and 14 (mwₐᵥₑ, 15,000) were obtained as lyophilized powder from the American Type Culture Collection (Rockville, Md.). Individual capsular polysaccharide preparations were dissolved in distilled H₂O at the concentration of 9 mg/ml and activated for 6 min with 1 mg of cyanogen bromide (Sigma Chemical Co., St. Louis, Mo.) at pH 10.5 and 4° C. After adjusting the pH to 8.5 with 0.2 M HCl, a solution of adipic acid dihydrazide in NaHCO₃ (0.5M) was added to the reaction mixture, to obtain a final carbohydrate concentration of 0.3 M. The reaction was allowed to proceed overnight with stirring at 8° C. and the materials were dialyzed against 0.2 M NaCl for 24 h. The solution was lyophilized and the resulting solid dissolved in distilled H₂O and desalted on a Biorad Bio-gel P2 column (Biorad Hercules, CA) equilibrated with distilled H₂O. Fractions containing significant amounts of the polysaccharide were pooled and lyophilized.

The pan DR binding peptide was coupled to the activated capsular polysaccharide hydrazide described above via carbodiimide-mediated condensation using 1-ethyl-3-dimethylaminopropyl carbodiimide (EDC) (Sigma). The pan DR binding synthetic peptide (7.4 μmol/ml) and activated carbohydrate (7 μmol/ml) were mixed in H₂O and the pH was adjusted to 4.9 with 0.2 M HCl. Solid EDC was added to a final concentration of 0.1M and the mixture was stirred at 4° C. for 3 h. Next the reaction milieu was dialyzed (mw cutoff, 4,500) overnight against 0.2 M NaCl followed by a second dialysis step against H₂O and the dialysate was subsequently lyophilized. These conjugated materials were separated from unbound pan DR binding peptide by chromatography using a CL-6B Sepharose column (3×100 cm) equilibrated in 0.2 M NaCl. Column eluates were tested for carbohydrate and protein content using the Schiff assay for carbohydrate and the Micro BCA Protein Assay Reagent Kit (Pierce Rockford, Ill.) for pan DR binding peptide content.

### Adjuvant Formulations and Immunizations

Immunogenicity of vaccine preparations was evaluated using formulations without adjuvants and formulations based on Complete and Incomplete Freund's Adjuvants (CFA/IFA). Groups of 3 C57BL/6 mice (Jackson-Laboratories, Bar Harbor Me.) at 8 to 10 wk of age were immunized subcutaneously at the base of the tail with 50, 5 or 0.5 μg of the experimental vaccines formulated in CFA in a volume of 100 μl and booster doses in IFA were administered 4 wk later. Mice were bled 4 wk following the primary immunization and 2 wk following the booster immunization. Immunization using 50 μg of non-conjugated capsular polysaccharides was used to document baseline immunogenicity of these materials.

Alhydrogel Al(OH)₃ (Superfos Biosector, Vedback, Denmark) was used as representative of a vaccine adjuvant suitable for use in humans. Experimental vaccine doses of 50, 5, and 0.5 kg, were each adsorbed to 250 μg of Al(OH)₃, and used to immunize mice.

Vaccines were administered in volumes of 100 μl, two or three times at 3-4 wk intervals by subcutaneous injection at the base of the tail. Blood samples were obtained by tail vein bleed prior to immunization and at monthly intervals.

In the case of CD4⁺-specific ELISPOT determinations, the pan DR binding peptide-Ps9V conjugate was emulsified in CFA and mice were immunized subcutaneously at the base of the tail with 100 μl (100 μg/mouse final). The pan DR binding peptide epitope (10 μg/mouse final) and Ps9V (50 μg/mouse final) controls were similarly prepared and used to immunize mice. Eleven to 14 days following immunization, the mice were sacrificed and the splenocytes were purified for ELISPOT measurements. All procedures were completed in a manner that was compliant with National Institutes of Health guidelines using Institutional Animal Care and Use Committee approved animal protocols.

### Measurement of Immune Responses

An ELISPOT assay was used to measure interferon-gamma (IFN-γ) production by CD4⁺ lymphocytes responding to the pan DR binding peptide (Tangri S, et al. J Exp Med 194(6):833-46 (2001)). Responses measured using the HCV core 28 epitope (GQIVGGVYLLPRRGPR (SEQ ID NO:7)) with splenocytes from immunized mice and responses of naïve mice to the pan DR binding peptide were used to establish background values. The t-test was used to determine significance of differences between means of triplicate tests.

Antibodies specific for pneumococcal polysaccharides were measured by ELISA (Yu X, et al. Clinical and Diagnostic Laboratory Immunology 6(4):519-24 (1999)).Total antibody levels and isotype specific responses were both measured. The opsonophagocytic killing assay, specific for the 6B serotype (strain DS2212) was used to assess antibody function associated with protective activity (Nahm M H, et al. Vaccine 18(24):2768-71 (2000)).

### Results

Immunogenicity of the Pan DR Binding Peptide in Experimental S. pneumoniae Capsular Polysaccharide Conjugate Vaccines

The integrity of the pan DR binding peptide following conjugation to S. pneumoniae capsular polysaccharides was evaluated as a function of immunogenicity. The CD4⁺ lymphocytes from mice immunized with the pan DR binding peptide-Ps9V conjugate emulsified in CFA/IFA responded specifically and vigorously to both the conjugate vaccine and the pan DR binding peptide. These data support the conclusion that the pan DR binding peptide is functional following conjugation using adipic dihydrazide chemistry to the S. pneumoniae capsular polysaccharide.

Antibody Responses to Pan DR Binding Peptide-S. pneumoniae Polysaccharide Conjugate Vaccines

The immunogenicity of the pan DR binding peptide-polysaccharide conjugate vaccines was first documented using CFA/IFA-based formulations. The non-conjugated polysaccharides were only poorly immunogenic whereas the conjugate forms proved to be potent immunogens. The potency of the products varied with serotype 14 being the most immunogenic and serotype 9 the least. The experimental vaccines were also evaluated in formulations consistent with human use, specifically PBS or Al(OH)₃. The serotype 14 product was equally immunogenic adsorbed onto Al(OH)₃ or administered in PBS whereas the Serotype 6B and 9V products were more immunogenic in the Al(OH)₃ formulation.

These data clearly demonstrate the increase in immunogenicity associated with conjugation of the polysaccharide to the pan DR binding peptide and its compatibility with formulation formats that can be used for human vaccine products.

### Requirement for Conjugation of the Pan DR Binding Peptide to Polysaccharides for Vaccine Carrier Effect

The pan DR binding peptide and free carbohydrates were adsorbed to Al(OH)₃. Conjugated forms of the same polysaccharides were mixed, adsorbed to Al(OH)₃ and used as the comparison immunogen. Antibodies specific to the polysaccharides were not detected until three immunizations of unconjugated pan DR binding peptide and polysaccharides with corresponding responses of relatively low titer, in the 200 to 1,000 range. In contrast, high-titered polysaccharide-specific antibody responses were induced using the same dose of polysaccharide conjugated to the pan DR binding peptide and adsorbed to Al(OH)₃. These data demonstrate an absolute requirement for conjugation of the pan DR binding peptide, the carrier component of the immunogen, and the polysaccharide, the B lymphocyte epitope component. It was also noted that the polysaccharides-pan DR binding peptide conjugate immunogens, administered in a multi-valent form, were equally immunogenic as when administered separately; they did not interfere or compete with each other to any significant degree.

### Antibody Isotype and Function

In the absence of an adjuvant, the experimental conjugate vaccines induced a predominance of IgG1 (37-82%) with significant contributions from IgM (44 and 58%) induced by pan DR binding peptide-Ps6B and pan DR binding peptide-Ps9V, respectively. Adsorption of the vaccines to Al(OH)₃ also resulted in IgG1 as the major isotype response observed. The only exception was the highly immunogenic pan DR binding peptide-Ps14, which induced significant amounts of IgG2b and IgG3, 10.6% and 6.8% of the total, respectively. Delivery of the experimental vaccines emulsified in CFA/IFA increased the relative amounts IgG2b (7.2 to 25.2%) and IgG3 (2.0 to 8.3%). Thus, IgG1 was the primary antibody isotype produced but the use of the potent CFA/IFA adjuvants altered responses.

The antibody isotype produced in response to immunization contributes to potential vaccine efficacy. For protection against S. pneumoniae, where the augmentation of phagocytosis and killing is needed, IgG1 should be a suitable isotype. To directly assess this, we measured phagocytosis and killing of S. pneumoniae strain 6B by granulocytes in the presence of dilutions of sera, from vaccinated mice, and complement using a common opsonophagocytosis assay. The opsonization titers correlated with total antibody titers; they were lowest in sera from mice immunized with unconjugated Ps6B but increased significantly when the experimental conjugate vaccine was used. An interesting observation was that the Al(OH)₃ proved to be the better suited adjuvant, inducing significantly higher opsonization titers that the CFA/IFA. These data demonstrate that antibodies induced by immunization with PADRE-Ps6B are functional and would be protective in vivo.

The above examples are provided to illustrate the invention but not to limit its scope. Other variants of the invention will be readily apparent to one of ordinary skill in the art and are encompassed by the appended claims. All publications, patents, and patent applications cited herein are hereby incorporated by reference.

1. A composition comprising a mixture of at least two Streptococcus pneumoniae capsular polysaccharides from different Streptococcus pneumoniae serotypes, wherein the capsular polysaccharide from each serotype is conjugated to a separate polypeptide comprising a pan DR binding peptide sequence independently selected from the formula R₁-R₂-R₃-R₄-R₅ (SEQ ID NOS:147-149), wherein:
R₁ is an amino acid followed by alanine or lysine;
R₂ is selected from the group consisting of tyrosine, phenylalanine or cyclohexylalanine;
R₃ is 3 or 4 amino acids, wherein each amino acid is independently selected from the group consisting of alanine, isoleucine, serine, glutamic acid and valine;
R₄ is selected from the group consisting of threonine-leucine-lysine, lysine-threonine, or tryptophan-threonine-leucine-lysine; and,
R₅ consists of 2 to 4 amino acids followed by an amino acid wherein each of the 2 to 4 amino acids is independently selected from the group consisting of alanine, serine, and valine.

2. The composition of claim 1, comprising capsular polysaccharides from at least any five of the following serotypes serotypes: 1, 2, 3, 4, 5, 6B, 7F, 8, 9N, 9V, 10A, 11A, 12F, 14, 15B, 17F, 18C, 19A, 19F, 20, 22F, 23F, 33F, 6A, 7A, 7B, 7C, 9A, 9L, 12A, 13, 15A, 15C, 16F, 18A, 18B, 18F, 19B, 19C, 21, 22A, 23A, 23B, 24F, 25, 27, 29, 31, 34, 35, 38, 45, or 46, wherein each polysaccharide is conjugated to a separate polypeptide comprising the pan DR binding peptide sequence.

3. The composition of claim 1, wherein the capsular polysaccharide is purified from bacteria of each serotype and conjugated to the polypeptide.

4. The composition of claim 3, wherein capsular polysaccharide from each serotype is separately conjugated to a polypeptide comprising the pan DR peptide and the resulting conjugates are subsequently combined to form a mixture of conjugates.

5. The composition of claim 3, wherein capsular polysaccharides from each serotype are combined to form a mixture of polysaccharides and the mixture is subsequently conjugated to polypeptides comprising the pan DR binding peptide.

6. The composition of claim 1, wherein a polypeptide comprising the pan DR binding peptide comprises the amino acid sequence AKXVAAWTLKAAA (SEQ ID NO:5), aKXVAAWTLKAAa, AKFVAAWTLKAAA (SEQ ID NO:6), or aKFVAAWTLKAAa, wherein X is cyclohexylalanine.

7. A method of making a Streptococcus pneumoniae vaccine, the method comprising,
conjugating at least two Streptococcus pneumoniae capsular polysaccharides from different Streptococcus pneumoniae serotypes to two separate polypeptides, each comprising a pan DR binding peptide sequence, wherein the pan DR binding peptide sequence is selected from the formula R₁-R₂-R₃-R₄-R₅ (SEQ ID NOS:147-149), wherein:
R₁ is an amino acid followed by alanine or lysine;
R₂ is selected from the group consisting of tyrosine, phenylalanine or cyclohexylalanine;
R₃ is 3 or 4 amino acids, wherein each amino acid is independently selected from the group consisting of alanine, isoleucine, serine, glutamic acid and valine;
R₄ is selected from the group consisting of threonine-leucine-lysine, lysine-threonine, or tryptophan-threonine-leucine-lysine (SEQ ID NO:150); and,
R₅ consists of 2 to 4 amino acids followed by an amino acid wherein each of the 2 to 4 amino acids is independently selected from the group consisting of alanine, serine, and valine.

8. The method of claim 7, comprising capsular polysaccharides from at least any five of the following serotypes serotypes: 1, 2, 3, 4, 5, 6B, 7F, 8, 9N, 9V, 10A, 11A, 12F, 14, 15B, 17F, 18C, 19A, 19F, 20, 22F, 23F, 33F, 6A, 7A, 7B, 7C, 9A, 9L, 12A, 13, 15A, 15C, 16F, 18A, 18B, 18F, 19B, 19C, 21, 22A, 23A, 23B, 24F, 25, 27, 29, 31, 34, 35, 38, 45, or 46, wherein each polysaccharide is conjugated to a separate polypeptide comprising the pan DR binding peptide sequence.

9. The method of claim 7, wherein the capsular polysaccharide from each serotype is separately conjugated to a polypeptide comprising the pan DR binding peptide and the resulting conjugates are subsequently combined to form a mixture of conjugates.

10. The method of claim 7, wherein capsular polysaccharides from each serotype are combined to form a mixture of polysaccharides and the mixture is subsequently conjugated to polypeptides comprising the pan DR binding peptide.

11. The method of claim 7, wherein the polypeptides each comprises the amino acid sequence AKXVAAWTLKAAA (SEQ ID NO:5), aKXVAAWTLKAAa, AKFVAAWTLKAAA (SEQ ID NO:6), or aKFVAAWTLKAAa, wherein X is cyclohexylalanine.

12. A method of inducing an immune response in a mammal, the method comprising,
administering to the mammal a mixture of at least two Streptococcus pneumoniae capsular polysaccharides from different Streptococcus pneumoniae serotypes, wherein the capsular polysaccharide from each serotype is conjugated to a separate pan DR binding peptide sequence selected from the formula R₁-R₂-R₃-R₄-R₅ (SEQ ID NOS:147-149), wherein:
R₁ is an amino acid followed by alanine or lysine;
R₂ is selected from the group consisting of tyrosine, phenylalanine or cyclohexylalanine;
R₃ is 3 or 4 amino acids, wherein each amino acid is independently selected from the group consisting of alanine, isoleucine, serine, glutamic acid and valine;
R₄ is selected from the group consisting of threonine-leucine-lysine, lysine-threonine, or tryptophan-threonine-leucine-lysine (SEQ ID NO:150); and,
R₅ consists of 2 to 4 amino acids followed by an amino acid wherein each of the 2 to 4 amino acids is independently selected from the group consisting of alanine, serine, and valine.

13. The method of claim 12, comprising capsular polysaccharides from at least any five of the following serotypes serotypes: 1, 2, 3, 4, 5, 6B, 7F, 8, 9N, 9V, 10A, 11A, 12F, 14, 15B, 17F, 18C, 19A, 19F, 20, 22F, 23F, 33F, 6A, 7A, 7B, 7C, 9A, 9L, 12A, 13, 15A, 15C, 16F, 18A, 18B, 18F, 19B, 19C, 21, 22A, 23A, 23B, 24F, 25, 27, 29, 31, 34, 35, 38, 45, or 46, wherein each polysaccharide is conjugated to a separate polypeptide comprising the pan DR binding peptide sequence.

14. The method of claim 12, wherein the capsular polysaccharide is purified from bacteria of the appropriate serotype and conjugated to the polypeptide.

15. The method of claim 12, wherein capsular polysaccharide from each serotype is separately conjugated to a polypeptide comprising the pan DR binding peptide and the resulting conjugates are subsequently combined to form a mixture of conjugates.

16. The method of claim 12, wherein capsular polysaccharides from each serotype are combined to form a mixture of polysaccharides and the mixture is subsequently conjugated to polypeptides comprising the pan DR binding peptide.

17. The method of claim 12, wherein a polypeptide comprising the pan DR binding peptide comprises the amino acid sequence AKXVAAWTLKAAA (SEQ ID NO:5), aKXVAAWTLKAAa, AKFVAAWTLKAAA (SEQ ID NO:6), or aKFVAAWTLKAAa, wherein X is cyclohexylalanine.


SEQUENCE LISTING (171 sequences)

SEQ ID NO: 1 - protein, 13 aa, Artificial Sequence
  pan DR binding peptide
AAFAAAKTAAAAA

SEQ ID NO: 2 - protein, 13 aa, Artificial Sequence
  pan DR binding peptide
AAFAAAATLKAAA

SEQ ID NO: 3 - protein, 13 aa, Artificial Sequence
  pan DR binding peptide
AAFVAAATLKAAA

SEQ ID NO: 4 - protein, 13 aa, Artificial Sequence
  pan DR binding peptide
AAFIAAATLKAAA

SEQ ID NO: 5 - protein, 13 aa, Artificial Sequence
  pan DR binding peptide
  MOD_RES (3)...(3): Xaa = cyclohexyalanine
AKXVAAWTLKAAA

SEQ ID NO: 6 - protein, 13 aa, Artificial Sequence
  pan DR binding peptide
AKFVAAWTLKAAA

SEQ ID NO: 7 - protein, 16 aa, Artificial Sequence
  HCV core 28 epitope
GQIVGGVYLLPRRGPR

SEQ ID NO: 8 - protein, 13 aa, Artificial Sequence
  pan DR binding peptide
  MOD_RES (3)...(3): Xaa = cyclohexylalanine
AKXVAAWTLKAAA

SEQ ID NO: 9 - protein, 13 aa, Artificial Sequence
  pan DR binding peptide
AKFVAAWTLKAAA

SEQ ID NO: 10 - protein, 13 aa, Artificial Sequence
  pan DR binding peptide
  MOD_RES (3)...(3): Xaa = cyclohexylalanine
AEXAAAATLKAAA

SEQ ID NO: 11 - skipped

SEQ ID NO: 12 - protein, 13 aa, Artificial Sequence
  pan DR binding peptide
  MOD_RES (3)...(3): Xaa = cyclohexylalanine
AQXAAAATLKAAA

SEQ ID NO: 13 - protein, 13 aa, Artificial Sequence
  pan DR binding peptide
  MOD_RES (3)...(3): Xaa = cyclohexylalanine
AVXAAAATLKAAA

SEQ ID NO: 14 - protein, 13 aa, Artificial Sequence
  pan DR binding peptide
  MOD_RES (3)...(3): Xaa = cyclohexylalanine
AFXAAAATLKAAA

SEQ ID NO: 15 - protein, 13 aa, Artificial Sequence
  pan DR binding peptide
  MOD_RES (3)...(3): Xaa = cyclohexylalanine
AAXKAAATLKAAA

SEQ ID NO: 16 - protein, 13 aa, Artificial Sequence
  pan DR binding peptide
  MOD_RES (3)...(3): Xaa = cyclohexylalanine
AAXEAAATLKAAA

SEQ ID NO: 17 - protein, 13 aa, Artificial Sequence
  pan DR binding peptide
  MOD_RES (3)...(3): Xaa = cyclohexylalanine
AAXQAAATLKAAA

SEQ ID NO: 18 - protein, 13 aa, Artificial Sequence
  pan DR binding peptide
  MOD_RES (3)...(3): Xaa = cyclohexylalanine
AAXFAAATLKAAA

SEQ ID NO: 19 - protein, 13 aa, Artificial Sequence
  pan DR binding peptide
  MOD_RES (3)...(3): Xaa = cyclohexylalanine
AAXLAAATLKAAA

SEQ ID NO: 20 - protein, 13 aa, Artificial Sequence
  pan DR binding peptide
  MOD_RES (3)...(3): Xaa = cyclohexylalanine
AAXAKAATLKAAA

SEQ ID NO: 21 - protein, 13 aa, Artificial Sequence
  pan DR binding peptide
  MOD_RES (3)...(3): Xaa = cyclohexylalanine
AAXAEAATLKAAA

SEQ ID NO: 22 - protein, 13 aa, Artificial Sequence
  pan DR binding peptide
  MOD_RES (3)...(3): Xaa = cyclohexylalanine
AAXAQAATLKAAA

SEQ ID NO: 23 - protein, 13 aa, Artificial Sequence
  pan DR binding peptide
  MOD_RES (3)...(3): Xaa = cyclohexylalanine
AAXAVAATLKAAA

SEQ ID NO: 24 - protein, 13 aa, Artificial Sequence
  pan DR binding peptide
  MOD_RES (3)...(3): Xaa = cyclohexylalanine
AAXAFAATLKAAA

SEQ ID NO: 25 - protein, 13 aa, Artificial Sequence
  pan DR binding peptide
  MOD_RES (3)...(3): Xaa = cyclohexylalanine
AAXAAKATLKAAA

SEQ ID NO: 26 - protein, 13 aa, Artificial Sequence
  pan DR binding peptide
  MOD_RES (3)...(3): Xaa = cyclohexylalanine
AAXAAEATLKAAA

SEQ ID NO: 27 - protein, 13 aa, Artificial Sequence
  pan DR binding peptide
  MOD_RES (3)...(3): Xaa = cyclohexylalanine
AAXAAQATLKAAA

SEQ ID NO: 28 - protein, 13 aa, Artificial Sequence
  pan DR binding peptide
  MOD_RES (3)...(3): Xaa = cyclohexylalanine
AAXAAVATLKAAA

SEQ ID NO: 29 - protein, 13 aa, Artificial Sequence
  pan DR binding peptide
  MOD_RES (3)...(3): Xaa = cyclohexylalanine
AAXAAFATLKAAA

SEQ ID NO: 30 - protein, 13 aa, Artificial Sequence
  pan DR binding peptide
  MOD_RES (3)...(3): Xaa = cyclohexylalanine
AAXAAAKTLKAAA

SEQ ID NO: 31 - protein, 13 aa, Artificial Sequence
  pan DR binding peptide
  MOD_RES (3)...(3): Xaa = cyclohexylalanine
AAXAAAETLKAAA

SEQ ID NO: 32 - protein, 13 aa, Artificial Sequence
  pan DR binding peptide
  MOD_RES (3)...(3): Xaa = cyclohexylalanine
AAXAAAQTLKAAA

SEQ ID NO: 33 - protein, 13 aa, Artificial Sequence
  pan DR binding peptide
  MOD_RES (3)...(3): Xaa = cyclohexylalanine
AAXAAAVTLKAAA

SEQ ID NO: 34 - protein, 13 aa, Artificial Sequence
  pan DR binding peptide
  MOD_RES (3)...(3): Xaa = cyclohexylalanine
AAXAAAFTLKAAA

SEQ ID NO: 35 - protein, 13 aa, Artificial Sequence
  pan DR binding peptide
  MOD_RES (3)...(3): Xaa = cyclohexylalanine
AAXAAATTLKAAA

SEQ ID NO: 36 - protein, 13 aa, Artificial Sequence
  pan DR binding peptide
  MOD_RES (3)...(3): Xaa = cyclohexylalanine
AAXAAAATKKAAA

SEQ ID NO: 37 - protein, 13 aa, Artificial Sequence
  pan DR binding peptide
  MOD_RES (3)...(3): Xaa = cyclohexylalanine
AAXAAAATEKAAA

SEQ ID NO: 38 - protein, 13 aa, Artificial Sequence
  pan DR binding peptide
  MOD_RES (3)...(3): Xaa = cyclohexylalanine
AAXAAAATQKAAA

SEQ ID NO: 39 - protein, 13 aa, Artificial Sequence
  pan DR binding peptide
  MOD_RES (3)...(3): Xaa = cyclohexylalanine
AAXAAAATVKAAA

SEQ ID NO: 40 - protein, 13 aa, Artificial Sequence
  pan DR binding peptide
  MOD_RES (3)...(3): Xaa = cyclohexylalanine
AAXAAAATFKAAA

SEQ ID NO: 41 - protein, 13 aa, Artificial Sequence
  pan DR binding peptide
  MOD_RES (3)...(3): Xaa = cyclohexylalanine
AAXAAAATIKAAA

SEQ ID NO: 42 - protein, 13 aa, Artificial Sequence
  pan DR binding peptide
  MOD_RES (3)...(3): Xaa = cyclohexylalanine
AAXAAAATLEAAA

SEQ ID NO: 43 - protein, 13 aa, Artificial Sequence
  pan DR binding peptide
  MOD_RES (3)...(3): Xaa = cyclohexylalanine
AAXAAAATLQAAA

SEQ ID NO: 44 - protein, 13 aa, Artificial Sequence
  pan DR binding peptide
  MOD_RES (3)...(3): Xaa = cyclohexylalanine
AAXAAAATLVAAA

SEQ ID NO: 45 - protein, 13 aa, Artificial Sequence
  pan DR binding peptide
  MOD_RES (3)...(3): Xaa = cyclohexylalanine
AAXAAAATLFAAA

SEQ ID NO: 46 - protein, 13 aa, Artificial Sequence
  pan DR binding peptide
  MOD_RES (3)...(3): Xaa = cyclohexylalanine
AAXAAAATLRAAA

SEQ ID NO: 47 - protein, 13 aa, Artificial Sequence
  pan DR binding peptide
  MOD_RES (3)...(3): Xaa = cyclohexylalanine
AAXAAAATLKKAA

SEQ ID NO: 48 - protein, 13 aa, Artificial Sequence
  pan DR binding peptide
  MOD_RES (3)...(3): Xaa = cyclohexylalanine
AAXAAAATLKEAA

SEQ ID NO: 49 - protein, 13 aa, Artificial Sequence
  pan DR binding peptide
  MOD_RES (3)...(3): Xaa = cyclohexylalanine
AAXAAAATLKQAA

SEQ ID NO: 50 - protein, 13 aa, Artificial Sequence
  pan DR binding peptide
  MOD_RES (3)...(3): Xaa = cyclohexylalanine
AAXAAAATLKVAA

SEQ ID NO: 51 - protein, 13 aa, Artificial Sequence
  pan DR binding peptide
  MOD_RES (3)...(3): Xaa = cyclohexylalanine
AAXAAAATLKFAA

SEQ ID NO: 52 - protein, 13 aa, Artificial Sequence
  pan DR binding peptide
  MOD_RES (3)...(3): Xaa = cyclohexylalanine
AAXAAAATLKIAA

SEQ ID NO: 53 - protein, 13 aa, Artificial Sequence
  pan DR binding peptide
  MOD_RES (3)...(3): Xaa = cyclohexylalanine
AAXAAAATLKAKA

SEQ ID NO: 54 - protein, 13 aa, Artificial Sequence
  pan DR binding peptide
  MOD_RES (3)...(3): Xaa = cyclohexylalanine
AAXAAAATLKAEA

SEQ ID NO: 55 - protein, 13 aa, Artificial Sequence
  pan DR binding peptide
  MOD_RES (3)...(3): Xaa = cyclohexylalanine
AAXAAAATLKAQA

SEQ ID NO: 56 - protein, 13 aa, Artificial Sequence
  pan DR binding peptide
  MOD_RES (3)...(3): Xaa = cyclohexylalanine
AAXAAAATLKAVA

SEQ ID NO: 57 - protein, 13 aa, Artificial Sequence
  pan DR binding peptide
  MOD_RES (3)...(3): Xaa = cyclohexylalanine
AAXAAAATLKAFA

SEQ ID NO: 58 - protein, 13 aa, Artificial Sequence
  pan DR binding peptide
  MOD_RES (3)...(3): Xaa = cyclohexylalanine
AKXVKANTLKAAA

SEQ ID NO: 59 - skipped

SEQ ID NO: 60 - protein, 13 aa, Artificial Sequence
  pan DR binding peptide
  MOD_RES (3)...(3): Xaa = cyclohexylalanine
AKXVKAWTLKAAA

SEQ ID NO: 61 - skipped

SEQ ID NO: 62 - protein, 13 aa, Artificial Sequence
  pan DR binding peptide
  MOD_RES (3)...(3): Xaa = cyclohexylalanine
AKXVWANTLKAAA

SEQ ID NO: 63 - protein, 13 aa, Artificial Sequence
  pan DR binding peptide
  MOD_RES (3)...(3): Xaa = cyclohexylalanine
AKXVWAYTLKAAA

SEQ ID NO: 64 - protein, 13 aa, Artificial Sequence
  pan DR binding peptide
  MOD_RES (3)...(3): Xaa = cyclohexylalanine
AKXVWAVTLKAAA

SEQ ID NO: 65 - protein, 13 aa, Artificial Sequence
  pan DR binding peptide
  MOD_RES (3)...(3): Xaa = cyclohexylalanine
AKXVYAWTLKAAA

SEQ ID NO: 66 - protein, 13 aa, Artificial Sequence
  pan DR binding peptide
  MOD_RES (3)...(3): Xaa = cyclohexylalanine
ARXVRANTLKAAA

SEQ ID NO: 67 - protein, 13 aa, Artificial Sequence
  pan DR binding peptide
  MOD_RES (3)...(3): Xaa = cyclohexylalanine
AKXVKAHTLKAAA

SEQ ID NO: 68 - skipped

SEQ ID NO: 69 - protein, 13 aa, Artificial Sequence
  pan DR binding peptide
  MOD_RES (3)...(3): Xaa = cyclohexylalanine
AKXVAANTLKAAA

SEQ ID NO: 70 - skipped

SEQ ID NO: 71 - protein, 13 aa, Artificial Sequence
  pan DR binding peptide
  MOD_RES (3)...(3): Xaa = cyclohexylalanine
AKXVAAYTLKAAA

SEQ ID NO: 72 - skipped

SEQ ID NO: 73 - protein, 13 aa, Artificial Sequence
  pan DR binding peptide
  MOD_RES (3)...(3): Xaa = cyclohexylalanine
AKXVAAWTLKAAA

SEQ ID NO: 74 - protein, 13 aa, Artificial Sequence
  pan DR binding peptide
  MOD_RES (3)...(3): Xaa = cyclohexylalanine
AKXVAAKTLKAAA

SEQ ID NO: 75 - protein, 13 aa, Artificial Sequence
  pan DR binding peptide
  MOD_RES (3)...(3): Xaa = cyclohexylalanine
AKXVAAHTLKAAA

SEQ ID NO: 76 - protein, 13 aa, Artificial Sequence
  pan DR binding peptide
  MOD_RES (3)...(3): Xaa = cyclohexylalanine
AKXVAAATLKAAA

SEQ ID NO: 77 - protein, 16 aa, Artificial Sequence
  pan DR binding peptide
  MOD_RES (6)...(6): Xaa = cyclohexylalanine
KSSAKXVMAATLKAAA

SEQ ID NO: 78 - protein, 13 aa, Artificial Sequence
  pan DR binding peptide
AKFAAAATLKAAA

SEQ ID NO: 79 - protein, 13 aa, Artificial Sequence
  pan DR binding peptide
AEFAAAATLKAAA

SEQ ID NO: 80 - skipped

SEQ ID NO: 81 - protein, 13 aa, Artificial Sequence
  pan DR binding peptide
AQFAAAATLKAAA

SEQ ID NO: 82 - protein, 13 aa, Artificial Sequence
  pan DR binding peptide
AVFAAAATLKAAA

SEQ ID NO: 83 - protein, 13 aa, Artificial Sequence
  pan DR binding peptide
AFFAAAATLKAAA

SEQ ID NO: 84 - protein, 13 aa, Artificial Sequence
  pan DR binding peptide
AAFKAAATLKAAA

SEQ ID NO: 85 - protein, 13 aa, Artificial Sequence
  pan DR binding peptide
AAFEAAATLKAAA

SEQ ID NO: 86 - protein, 13 aa, Artificial Sequence
  pan DR binding peptide
AAFQAAATLKAAA

SEQ ID NO: 87 - protein, 13 aa, Artificial Sequence
  pan DR binding peptide
AAFFAAATLKAAA

SEQ ID NO: 88 - protein, 13 aa, Artificial Sequence
  pan DR binding peptide
AAFLAAATLKAAA

SEQ ID NO: 89 - protein, 13 aa, Artificial Sequence
  pan DR binding peptide
AAFAKAATLKAAA

SEQ ID NO: 90 - protein, 13 aa, Artificial Sequence
  pan DR binding peptide
AAFAEAATLKAAA

SEQ ID NO: 91 - protein, 13 aa, Artificial Sequence
  pan DR binding peptide
AAFAQAATLKAAA

SEQ ID NO: 92 - protein, 13 aa, Artificial Sequence
  pan DR binding peptide
AAFAVAATLKAAA

SEQ ID NO: 93 - protein, 13 aa, Artificial Sequence
  pan DR binding peptide
AAFAFAATLKAAA

SEQ ID NO: 94 - protein, 13 aa, Artificial Sequence
  pan DR binding peptide
AAFAAKATLKAAA

SEQ ID NO: 95 - protein, 13 aa, Artificial Sequence
  pan DR binding peptide
AAFAAEATLKAAA

SEQ ID NO: 96 - protein, 13 aa, Artificial Sequence
  pan DR binding peptide
AAFAAQATLKAAA

SEQ ID NO: 97 - protein, 13 aa, Artificial Sequence
  pan DR binding peptide
AAFAAVATLKAAA

SEQ ID NO: 98 - protein, 13 aa, Artificial Sequence
  pan DR binding peptide
AAFAAFATLKAAA

SEQ ID NO: 99 - protein, 13 aa, Artificial Sequence
  pan DR binding peptide
AAFAAAKTLKAAA

SEQ ID NO: 100 - protein, 13 aa, Artificial Sequence
  pan DR binding peptide
AAFAAAETLKAAA

SEQ ID NO: 101 - protein, 13 aa, Artificial Sequence
  pan DR binding peptide
AAFAAAQTLKAAA

SEQ ID NO: 102 - protein, 13 aa, Artificial Sequence
  pan DR binding peptide
AAFAAAVTLKAAA

SEQ ID NO: 103 - protein, 13 aa, Artificial Sequence
  pan DR binding peptide
AAFAAAFTLKAAA

SEQ ID NO: 104 - protein, 13 aa, Artificial Sequence
  pan DR binding peptide
AAFAAATTLKAAA

SEQ ID NO: 105 - protein, 13 aa, Artificial Sequence
  pan DR binding peptide
AAFAAAATKKAAA

SEQ ID NO: 106 - protein, 13 aa, Artificial Sequence
  pan DR binding peptide
AAFAAAATEKAAA

SEQ ID NO: 107 - protein, 13 aa, Artificial Sequence
  pan DR binding peptide
AAFAAAATQKAAA

SEQ ID NO: 108 - protein, 13 aa, Artificial Sequence
  pan DR binding peptide
AAFAAAATVKAAA

SEQ ID NO: 109 - protein, 13 aa, Artificial Sequence
  pan DR binding peptide
AAFAAAATFKAAA

SEQ ID NO: 110 - protein, 13 aa, Artificial Sequence
  pan DR binding peptide
AAFAAAATIKAAA

SEQ ID NO: 111 - protein, 13 aa, Artificial Sequence
  pan DR binding peptide
AAFAAAATLEAAA

SEQ ID NO: 112 - protein, 13 aa, Artificial Sequence
  pan DR binding peptide
AAFAAAATLQAAA

SEQ ID NO: 113 - protein, 13 aa, Artificial Sequence
  pan DR binding peptide
AAFAAAATLVAAA

SEQ ID NO: 114 - protein, 13 aa, Artificial Sequence
  pan DR binding peptide
AAFAAAATLFAAA

SEQ ID NO: 115 - protein, 13 aa, Artificial Sequence
  pan DR binding peptide
AAFAAAATLRAAA

SEQ ID NO: 116 - protein, 13 aa, Artificial Sequence
  pan DR binding peptide
AAFAAAATLKKAA

SEQ ID NO: 117 - protein, 13 aa, Artificial Sequence
  pan DR binding peptide
AAFAAAATLKEAA

SEQ ID NO: 118 - protein, 13 aa, Artificial Sequence
  pan DR binding peptide
AAFAAAATLKQAA

SEQ ID NO: 119 - protein, 13 aa, Artificial Sequence
  pan DR binding peptide
AAFAAAATLKVAA

SEQ ID NO: 120 - protein, 13 aa, Artificial Sequence
  pan DR binding peptide
AAFAAAATLKFAA

SEQ ID NO: 121 - protein, 13 aa, Artificial Sequence
  pan DR binding peptide
AAFAAAATLKIAA

SEQ ID NO: 122 - protein, 13 aa, Artificial Sequence
  pan DR binding peptide
AAFAAAATLKAKA

SEQ ID NO: 123 - protein, 13 aa, Artificial Sequence
  pan DR binding peptide
AAFAAAATLKAEA

SEQ ID NO: 124 - protein, 13 aa, Artificial Sequence
  pan DR binding peptide
AAFAAAATLKAQA

SEQ ID NO: 125 - protein, 13 aa, Artificial Sequence
  pan DR binding peptide
AAFAAAATLKAVA

SEQ ID NO: 126 - protein, 13 aa, Artificial Sequence
  pan DR binding peptide
AAFAAAATLKAFA

SEQ ID NO: 127 - protein, 13 aa, Artificial Sequence
  pan DR binding peptide
AKFVKANTLKAAA

SEQ ID NO: 128 - protein, 13 aa, Artificial Sequence
  pan DR binding peptide
AKFVKANTLKAAA

SEQ ID NO: 129 - protein, 13 aa, Artificial Sequence
  pan DR binding peptide
AKFVKAWTLKAAA

SEQ ID NO: 130 - protein, 13 aa, Artificial Sequence
  pan DR binding peptide
AKFVKAWTLKAAA

SEQ ID NO: 131 - protein, 13 aa, Artificial Sequence
  pan DR binding peptide
AKFVWANTLKAAA

SEQ ID NO: 132 - protein, 13 aa, Artificial Sequence
  pan DR binding peptide
AKFVWAYTLKAAA

SEQ ID NO: 133 - protein, 13 aa, Artificial Sequence
  pan DR binding peptide
AKFVWAVTLKAAA

SEQ ID NO: 134 - protein, 13 aa, Artificial Sequence
  pan DR binding peptide
AKFVYAWTLKAAA

SEQ ID NO: 135 - protein, 13 aa, Artificial Sequence
  pan DR binding peptide
ARFVRANTLKAAA

SEQ ID NO: 136 - protein, 13 aa, Artificial Sequence
  pan DR binding peptide
AKFVKAHTLKAAA

SEQ ID NO: 137 - skipped

SEQ ID NO: 138 - protein, 13 aa, Artificial Sequence
  pan DR binding peptide
AKFVAANTLKAAA

SEQ ID NO: 139 - skipped

SEQ ID NO: 140 - protein, 13 aa, Artificial Sequence
  pan DR binding peptide
AKFVAAYTLKAAA

SEQ ID NO: 141 - skipped

SEQ ID NO: 142 - protein, 13 aa, Artificial Sequence
  pan DR binding peptide
AKFVAAWTLKAAA

SEQ ID NO: 143 - protein, 13 aa, Artificial Sequence
  pan DR binding peptide
AKFVAAKTLKAAA

SEQ ID NO: 144 - protein, 13 aa, Artificial Sequence
  pan DR binding peptide
AKFVAAHTLKAAA

SEQ ID NO: 145 - protein, 13 aa, Artificial Sequence
  pan DR binding peptide
AKFVAAATLKAAA

SEQ ID NO: 146 - protein, 16 aa, Artificial Sequence
  pan DR binding peptide
KSSAKFVMAATLKAAA

SEQ ID NO: 147 - protein, 14 aa, Artificial Sequence
  pan DR binding peptide
  MOD_RES (1)...(1): Xaa = any amino acid
  MOD_RES (2)...(2): Xaa = Ala or Lys
  MOD_RES (3)...(3): Xaa = Tyr, Phe or cyclohexylalanine
  MOD_RES (4)...(6): Xaa = Ala, Ile, Ser, Glu or Val
  MOD_RES (7)...(7): Xaa = Ala, Ile, Ser, Glu or Val, Xaa at position 7 may be present or absent
  MOD_RES (11)...(12): Xaa = Ala, Ser or Val
  MOD_RES (13)...(14): Xaa = Ala, Ser or Val, Xaa at positions 13 and 14 may be present or absent
XXXXXXXTLKXXXX

SEQ ID NO: 148 - protein, 13 aa, Artificial Sequence
  pan DR binding peptide
  MOD_RES (1)...(1): Xaa = any amino acid
  MOD_RES (2)...(2): Xaa = Ala or Lys
  MOD_RES (3)...(3): Xaa = Tyr, Phe or cyclohexylalanine
  MOD_RES (4)...(6): Xaa = Ala, Ile, Ser, Glu or Val
  MOD_RES (7)...(7): Xaa = Ala, Ile, Ser, Glu or Val, Xaa at position 7 may be present or absent
  MOD_RES (10)...(11): Xaa = Ala, Ser or Val
  MOD_RES (12)...(13): Xaa = Ala, Ser or Val, Xaa at positions 13 and 14 may be present or absent
XXXXXXXKTXXXX

SEQ ID NO: 149 - protein, 15 aa, Artificial Sequence
  pan DR binding peptide
  MOD_RES (1)...(1): Xaa = any amino acid
  MOD_RES (2)...(2): Xaa = Ala or Lys
  MOD_RES (3)...(3): Xaa = Tyr, Phe or cyclohexylalanine
  MOD_RES (4)...(6): Xaa = Ala, Ile, Ser, Glu or Val
  MOD_RES (7)...(7): Xaa = Ala, Ile, Ser, Glu or Val, Xaa at position 7 may be present or absent
  MOD_RES (12)...(13): Xaa = Ala, Ser or Val
  MOD_RES (14)...(15): Xaa = Ala, Ser or Val, Xaa at positions 13 and 14 may be present or absent
XXXXXXXWTLKXXXX

SEQ ID NO: 150 - protein, 4 aa, Artificial Sequence
  optional R4 in pan DR binding peptide formula R1-R2-R3-R4-R5
WTLK

SEQ ID NO: 151 - protein, 13 aa, Artificial Sequence
  pan DR binding peptide
  MOD_RES (3)...(3): Xaa = cyclohexylalanine
AAXAAAKTAAAAA

SEQ ID NO: 152 - protein, 13 aa, Artificial Sequence
  pan DR binding peptide
  MOD_RES (3)...(3): Xaa = cyclohexylalanine
AAXAAAATLKAAA

SEQ ID NO: 153 - protein, 13 aa, Artificial Sequence
  pan DR binding peptide
  MOD_RES (3)...(3): Xaa = cyclohexylalanine
AAXVAAATLKAAA

SEQ ID NO: 154 - protein, 13 aa, Artificial Sequence
  pan DR binding peptide
  MOD_RES (3)...(3): Xaa = cyclohexylalanine
AAXIAAATLKAAA

SEQ ID NO: 155 - protein, 13 aa, Artificial Sequence
  pan DR binding peptide
  MOD_RES (1)...(2): Xaa = any amino acid
  MOD_RES (3)...(3): Xaa = Tyr, Phe or cyclohexylalanine
  MOD_RES (4)...(6): Xaa = any amino acid
  MOD_RES (7)...(8): Xaa = any amino acid, Xaa at positions 7 and 8 may be present or absent
  MOD_RES (12)...(13): Xaa = any amino acid
XXXXXXXXTLKXX

SEQ ID NO: 156 - protein, 12 aa, Artificial Sequence
  pan DR binding peptide
  MOD_RES (1)...(2): Xaa = any amino acid
  MOD_RES (3)...(3): Xaa = Tyr, Phe or cyclohexylalanine
  MOD_RES (4)...(6): Xaa = any amino acid
  MOD_RES (7)...(8): Xaa = any amino acid, Xaa at positions 7 and 8 may be present or absent
  MOD_RES (11)...(12): Xaa = any amino acid
XXXXXXXXKTXX

SEQ ID NO: 157 - protein, 14 aa, Artificial Sequence
  pan DR binding peptide
  MOD_RES (1)...(2): Xaa = any amino acid
  MOD_RES (3)...(3): Xaa = Tyr, Phe or cyclohexylalanine
  MOD_RES (4)...(6): Xaa = any amino acid
  MOD_RES (7)...(8): Xaa = any amino acid, Xaa at positions 7 and 8 may be present or absent
  MOD_RES (13)...(14): Xaa = any amino acid
XXXXXXXXWTLKXX

SEQ ID NO: 158 - protein, 13 aa, Artificial Sequence
  pan DR binding peptide
  MOD_RES (3)...(3): Xaa = cyclohexylalanine
AKXAAAATLKAAA

SEQ ID NO: 159 - protein, 13 aa, Artificial Sequence
  Streptococcus pneumoniae polysaccharide 6B/pan DR binding peptide SEQ ID NO:6 conjugate
  MOD_RES (1)...(1): Xaa = Ala conjuagated to Streptococcus pneumoniae polysaccharide 6B
XKFVAAWTLKAAA

SEQ ID NO: 160 - protein, 13 aa, Artificial Sequence
  Streptococcus pneumoniae polysaccharide 4/pan DR binding peptide SEQ ID NO:6 conjugate
  MOD_RES (1)...(1): Xaa = Ala conjugated to Streptococcus pneumoniae polysaccharide 4
XKFVAAWTLKAAA

SEQ ID NO: 161 - protein, 13 aa, Artificial Sequence
  Streptococcus pneumoniae polysaccharide 9V/pan DR binding peptide SEQ ID NO:6 conjugate
  MOD_RES (1)...(1): Xaa = Ala conjugated to Streptococcus pneumoniae polysaccharide 9V
XKFVAAWTLKAAA

SEQ ID NO: 162 - protein, 13 aa, Artificial Sequence
  Streptococcus pneumoniae polysaccharide 14/pan DR binding peptide SEQ ID NO:6 conjugate
  MOD_RES (1)...(1): Xaa = Ala conjugated to Streptococcus pneumoniae polysaccharide 14
XKFVAAWTLKAAA

SEQ ID NO: 163 - protein, 13 aa, Artificial Sequence
  Streptococcus pneumoniae polysaccharide 18C/pan DR binding peptide SEQ ID NO:6 conjugate
  MOD_RES (1)...(1): Xaa = Ala conjugated to Streptococcus pneumoniae polysaccharide 18C
XKFVAAWTLKAAA

SEQ ID NO: 164 - protein, 13 aa, Artificial Sequence
  Streptococcus pneumoniae polysaccharide 19F/pan DR binding peptide SEQ ID NO:6 conjugate
  MOD_RES (1)...(1): Xaa = Ala conjugated to Streptococcus pneumoniae polysaccharide 19F
XKFVAAWTLKAAA

SEQ ID NO: 165 - protein, 13 aa, Artificial Sequence
  Streptococcus pneumoniae polysaccharide 23F/pan DR binding peptide SEQ ID NO:6 conjugate
  MOD_RES (1)...(1): Xaa = Ala conjugated to Streptococcus pneumoniae polysaccharide 23F
XKFVAAWTLKAAA

SEQ ID NO: 166 - protein, 13 aa, Artificial Sequence
  Streptococcus pneumoniae polysaccharide 4/pan DR binding peptide SEQ ID NO:5 conjugate
  MOD_RES (1)...(1): Xaa = Ala conjugated to Streptococcus pneumoniae polysaccharide 4
  MOD_RES (3)...(3): Xaa = cyclohexylalanine
XKXVAAWTLKAAA

SEQ ID NO: 167 - protein, 13 aa, Artificial Sequence
  Streptococcus pneumoniae polysaccharide 9V/pan DR binding peptide SEQ ID NO:5 conjugate
  MOD_RES (1)...(1): Xaa = Ala conjugated to Streptococcus pneumoniae polysaccharide 9V
  MOD_RES (3)...(3): Xaa = cyclohexylalanine
XKXVAAWTLKAAA

SEQ ID NO: 168 - protein, 13 aa, Artificial Sequence
  Streptococcus pneumoniae polysaccharide 14/pan DR binding peptide SEQ ID NO:5 conjugate
  MOD_RES (1)...(1): Xaa = Ala conjugated to Streptococcus pneumoniae polysaccharide 14
  MOD_RES (3)...(3): Xaa = cyclohexylalanine
XKXVAAWTLKAAA

SEQ ID NO: 169 - protein, 13 aa, Artificial Sequence
  Streptococcus pneumoniae polysaccharide 18C/pan DR binding peptide SEQ ID NO:5 conjugate
  MOD_RES (1)...(1): Xaa = Ala conjugated to Streptococcus pneumoniae polysaccharide 18C
  MOD_RES (3)...(3): Xaa = cyclohexylalanine
XKXVAAWTLKAAA

SEQ ID NO: 170 - protein, 13 aa, Artificial Sequence
  Streptococcus pneumoniae polysaccharide 19F/pan DR binding peptide SEQ ID NO:5 conjugate
  MOD_RES (1)...(1): Xaa = Ala conjugated to Streptococcus pneumoniae polysaccharide 19F
  MOD_RES (3)...(3): Xaa = cyclohexylalanine
XKXVAAWTLKAAA

SEQ ID NO: 171 - protein, 13 aa, Artificial Sequence
  Streptococcus pneumoniae polysaccharide 23F/pan DR binding peptide SEQ ID NO:5 conjugate
  MOD_RES (1)...(1): Xaa = Ala conjugated to Streptococcus pneumoniae polysaccharide 23F
  MOD_RES (3)...(3): Xaa = cyclohexylalanine
XKXVAAWTLKAAA